(19) United States
(12) Patent Application Publication
(10) Pub. No.: US 2013/0079670 A1
(43) Pub. Date: Mar. 28, 2013

(54) SELF-CONTAINED MUSCULAR-SKELETAL PARAMETER MEASUREMENT SYSTEM HAVING UN-LOADED OR LIGHTLY LOADED CAVITY

(71) Applicants: Marc Stein, Chandler, AZ (US); Andrew P. Miller, Gilbert, AZ (US); Jason Addink, Gilbert, AZ (US)

(72) Inventors: Marc Stein, Chandler, AZ (US); Andrew P. Miller, Gilbert, AZ (US); Jason Addink, Gilbert, AZ (US)

(73) Assignee: ORTHOSENSOR, Sunrise, FL (US)

(21) Appl. No.: 13/242,764

(22) Filed: Sep. 23, 2011

(51) Int. Cl. A61B 5/103

(52) U.S. Cl. 600/587

(57) ABSTRACT

At least one embodiment is directed to an insert sensing device for measuring a parameter of the muscular-skeletal system. The insert sensing device can be temporary or permanent. The insert sensing device is a self-contained encapsulated measurement device. The insert sensing device comprises a support structure having an articular surface for allowing articulation of the muscular-skeletal system and a support structure having a load bearing surface. The structures attach together to form a housing that includes one or more sensors, a power source, electronic circuitry, and communication circuitry. The electronic circuitry, power source, and communication circuitry are placed in a cavity of the insert sensing device that is unloaded or lightly loaded by the muscular-skeletal system. Shims can be attached to the load-bearing surface to adjust the height of insert sensing device.

### FIELD

The present invention pertains generally to a joint prosthesis, and particularly to methods and devices for assessing and determining proper loading and balance of an implant component or components during joint reconstructive surgery and long-term monitoring of the muscular-skeletal system.

### BACKGROUND

The skeletal system of a mammal is subject to variations among species. Further changes can occur due to environmental factors, degradation through use, and aging. An orthopedic joint of the skeletal system typically comprises two or more bones that move in relation to one another. Movement is enabled by muscle tissue and tendons attached to the skeletal system of the joint. Ligaments hold and stabilize the one or more joint bones positionally. Cartilage is a wear surface that prevents bone-to-bone contact, distributes load, and lowers friction.

There has been substantial growth in the repair of the human skeletal system. In general, prosthetic orthopedic joints have evolved using information from simulations, mechanical prototypes, and patient data that is collected and used to initiate improved designs. Similarly, the tools being used for orthopedic surgery have been refined over the years but have not changed substantially. Thus, the basic procedure for replacement of an orthopedic joint has been standardized to meet the general needs of a wide distribution of the population. Although the tools, procedure, and artificial joint meet a general need, each replacement procedure is subject to significant variation from patient to patient. The correction of these individual variations relies on the skill of the surgeon to adapt and fit the replacement joint using the available tools to the specific circumstance.

### BRIEF DESCRIPTION OF THE DRAWINGS

Various features of the system are set forth with particularity in the appended claims. The embodiments herein, can be understood by reference to the following description, taken in conjunction with the accompanying drawings, in which:

FIG. 1 illustrates an insert for measuring a parameter of the muscular-skeletal system in accordance with an example embodiment;

FIG. 2 illustrates an application of an insert sensing device in accordance with an example embodiment;

FIG. 3 illustrates the insert sensing device placed in a joint of the muscular-skeletal system for measuring a parameter in accordance with an example embodiment;

FIG. 4 illustrates an adjustable height insert sensing device in accordance with an example embodiment;

FIG. 5 illustrates an insert sensing device comprising a housing and a plurality of shims in accordance with an example embodiment;

FIG. 6 illustrates a lower support structure of an insert sensing device in accordance with an example embodiment;

FIG. 7 illustrates the lower support structure with the sensors located in cavities in accordance with an example embodiment;

FIG. 8 illustrates a plurality of load plates in accordance with an example embodiment;

FIG. 9 illustrates the lower support structure and the upper support structure in accordance with an example embodiment;

FIG. 10 illustrates attached components for an insert sensing device in accordance with an example embodiment;

FIG. 11 illustrates components of an insert sensing device in accordance with an example embodiment;

FIG. 12 illustrates a slot in insert sensing device in accordance with an example embodiment;

FIG. 13 illustrates the sensing module interfacing with the lower support structure in accordance with an example embodiment;

FIG. 14 is an example block diagram of the components of an insert sensing device in accordance with an example embodiment;

FIG. 15 illustrates a communications system for short-range telemetry in accordance with an example embodiment;

FIG. 16 illustrates a communication network for measurement and reporting in accordance with an example embodiment; and

FIG. 17 depicts an exemplary diagrammatic representation of a machine in the form of a computer system within which a set of instructions, when executed, may cause the machine to perform any one or more of the methodologies disclosed herein.

### DETAILED DESCRIPTION

Embodiments of the invention are broadly directed to measurement of physical parameters. More specifically, an electro-mechanical system is directed towards the measurement of parameters related to the muscular-skeletal system. Many physical parameters of interest within physical systems or bodies are currently not measured due to size, cost, time, or measurement precision. For example, joint implants such as knee, hip, spine, shoulder, and ankle implants would benefit substantially from in-situ measurements taken during surgery to aid the surgeon in fine-tuning the prosthetic system. Measurements can supplement the subjective feedback of the surgeon to ensure optimal installation. Permanent sensors in the final prosthetic components can provide periodic data related to the status of the implant in use. Data collected intra-operatively and long term can be used to determine parameter ranges for surgical installation and to improve future prosthetic components.

The physical parameter or parameters of interest can include, but are not limited to, measurement of load, force, pressure, position, displacement, density, viscosity, pH, spurious accelerations, and localized temperature. Often, a measured parameter is used in conjunction with another measured parameter to make a qualitative assessment. In joint reconstruction, portions of the muscular-skeletal system are prepared to receive prosthetic components. Preparation includes bone cuts or bone shaping to mate with one or more prosthesis. Parameters can be evaluated relative to orientation, alignment, direction, displacement, or position as well as movement, rotation, or acceleration along an axis or combination of axes by wireless sensing modules or devices positioned on or within a body, instrument, appliance, vehicle, equipment, or other physical system.

In all of the examples illustrated and discussed herein, any specific materials, such as temperatures, times, energies, and material properties for process steps or specific structure implementations should be interpreted to be illustrative only and non-limiting. Processes, techniques, apparatus, and materials as known by one of ordinary skill in the art may not be discussed in detail but are intended to be part of an enabling description where appropriate. It should also be noted that the word “coupled” used herein implies that elements may be directly coupled together or may be coupled through one or more intervening elements.

Note that similar reference numerals and letters refer to similar items in the following figures. In some cases, numbers from prior illustrations will not be placed on subsequent figures for purposes of clarity. In general, it should be assumed that structures not identified in a figure are the same as previous prior figures.

In the present invention parameters can be measured with an integrated wireless sensing module or device comprising an i) encapsulating structure that supports sensors and contacting surfaces and ii) an electronic assemblage that integrates a power supply, sensing elements, biasing spring or springs or other form of elastic members, an accelerometer, antennas and electronic circuitry that processes measurement data as well as controls all operations of energy conversion, propagation, and detection and wireless communications. The wireless sensing module or device can be positioned on or within, or engaged with, or attached or affixed to or within, a wide range of physical systems including, but not limited to instruments, appliances, vehicles, equipments, or other physical systems as well as animal and human bodies, for sensing and communicating parameters of interest in real time.

Embodiments of the invention are broadly directed to measurement of physical parameters. Sensors can measure many physical parameters of interest within physical systems or bodies. The sensors evaluate changes in the characteristics of the parameter being measured. As one example, changes in the transit time or shape of an energy wave or pulse propagating through a medium that is modified by a parameter can be measured and correlated to the parameter to produce a measurement. Alternatively, sensors can be used that directly measure the parameter such as a piezo-resistive film sensor that outputs a signal relative to a pressure applied thereto. The measurement system has a form factor, power usage, and material that is compatible with human body dynamics. The physical parameter or parameters of interest can include, but are not limited to, measurement of load, force, pressure, displacement, density, viscosity, pH, distance, volume, pain, infection, spurious acceleration, and localized temperature to name a few. These parameters can be evaluated by sensor measurement, alignment, direction, or position as well as movement, rotation, or acceleration along an axis or combination of axes by wireless sensing modules or devices positioned on or within a body, instrument, appliance, vehicle, equipment, or other physical system.

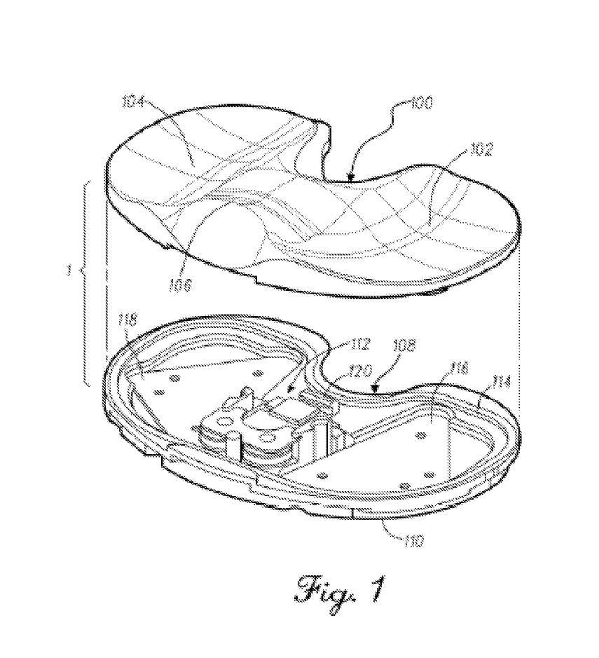

FIG. 1 illustrates an insert 1 for measuring a parameter of the muscular-skeletal system in accordance with an example embodiment. In general, a prosthetic insert is a component of a joint replacement system that allows articulation of the muscular-skeletal system. The prosthetic insert is a wear component of the joint replacement system. The prosthetic insert has one or more articular surfaces that allow joint articulation. In a joint replacement, a prosthetic component has a surface that couples to the articular surface of the insert. The articular surface is low friction and can absorb loading that occurs naturally based on situation or position. The contact area between surfaces of the articulating joint can vary over the range of motion. The articular surface of the insert will wear over time due to friction produced by the prosthetic component surface contacting the articular surface during movement of the joint. Ligaments, muscle, and tendons hold the joint together and motivate the joint throughout the range of motion.

Insert 1 is an active device which can have a power source, electronic circuitry, transmit capability, and sensors within the body of the prosthetic component. In one embodiment, insert 1 is used intra-operatively to measure parameters of the muscular-skeletal system to aid in the installation of one or more prosthetic components. As will be disclosed hereinbelow, operation of insert 1 is shown as a knee insert to illustrate operation and measurement of a parameter such as loading and balance. Insert 1 can be adapted for use in other prosthetic joints having articular surfaces such as the hip, spine, shoulder, ankle, and others. Alternatively, insert 1 can be a permanent active device that can be used to take parameter measurements over the life of the implant.

In both embodiments, insert 1 is substantially equal in dimensions to a passive final prosthetic insert. In general, the substantially equal dimensions correspond to size and shape that allow insert 1 to fit substantially equal to the passive final prosthetic insert. In the intra-operative example, the measured loading and balance using insert 1 as a trial insert would be substantially equal to the loading and balance seen by the final insert under equal conditions. It should be noted that insert 1 for intra-operative measurement could be dissimilar in shape or have missing features that do not benefit the trial during operation. Insert 1 should be positionally stable throughout the range of motion equal to that of the final insert.

The exterior structure of insert 1 is formed from at least two components. In the embodiment shown, insert 1 comprises a support structure 100 and a support structure 108. Support structures 100 and 108 have major support surfaces that are loaded by the muscular-skeletal system. As previously mentioned, insert 1 is shown as a knee insert to illustrate general concepts and is not limited to this configuration. Support structure 100 has an articular surface 102 and an articular surface 104. Condyles of a femoral prosthetic component articulate with surfaces 102 and 104. Loading on the prosthetic knee joint is distributed over a contact area of the articular surfaces 102 and 104. In general, accelerated wear occurs if the contact area is insufficient to support the load. A region 106 of the support structure 100 is unloaded or is lightly loaded over the range of motion. Region 106 is between the articular surfaces 102 and 104. It should be noted that there is an optimal area of contact on the articular surfaces to minimize wear while maintaining joint performance. The contact location can vary depending on the position of the muscular-skeletal system. Problems may occur if the contact area falls outside a predetermined area range within articular surfaces 102 and 104 over the range of motion. In one embodiment, the location where the load is applied on articular surfaces 102 and 104 is determined by the sensing system. This is beneficial because the surgeon now has quantitative information where the loading is applied. The surgeon can then make adjustments that move the location of the applied load within the predetermined area using real-time feedback from the sensing system to track the result of each correction.

The support structure 108 includes sensors and electronic circuitry 112 to measure loading on each articular surface of insert 1. A load plate 116 underlies articular surface 102. Similarly, a load plate 118 underlies articular surface 104. Force, pressure, or load sensors (not shown) underlie load plates 116 and 118. In one embodiment, load plates 116 and 118 distribute the load to a plurality of sensors for determining a location where the load is applied. Although the surface of load plates 116 and 118 as illustrated, are planar they can be conformal to the shape of an articular surface. A force, pressure, or load applied to articular surfaces 102 and 104 is respectively coupled to plates 116 and 118. Electronic circuitry 112 is operatively coupled to the sensors underlying load plates 116 and 118. Plates 116 and 118 distribute and couple a force, pressure, or load applied to the articular surface to the sensors. The sensors output signals corresponding to the force, pressure, or load applied to the articular surfaces, which are received and translated by electronic circuitry 112. The measurement data can be processed and transmitted to a receiver external to insert 1 for display and analysis. In one embodiment, the physical location of electronic circuitry 112 is located between articular surfaces 102 and 104, which correspond to region 106 of support structure 100. A cavity for housing the electronic circuitry 112 underlies region 106. Support structure 108 has a surface within the cavity having retaining features extending therefrom to locate and retain electronic circuitry 112 within the cavity. The retaining features are disclosed in more detail hereinbelow. This location is an unloaded or a lightly loaded region of insert 1 thereby reducing a potential of damaging the electronic circuitry 112 due to a compressive force during surgery or as the joint is used by the patient. In one embodiment, a temporary power source such as a battery, capacitor, inductor, or other storage medium is located within the insert to power the sensors and electronic circuitry 112.

Support structure 100 attaches to support structure 108 to form the insert casing. Internal surfaces of support structures 100 and 108 mate together. Moreover, the internal surfaces of support structures 100 and 108 can have cavities or extrusions to house and retain components of the sensing system. Externally, support structures 100 and 108 provide load bearing and articular surfaces that interface to the other prosthetic components of the joint. The support structure 108 has a support surface 110 that couples to a tibial implant. In general, the support surface 110 has a much greater load distributing surface area that reduces the force, pressure, or load per unit area than the articulating contact region of articular surfaces 102 and 104.

The support structures 100 and 108 can be temporarily or permanently coupled, attached, or fastened together. As shown, insert 1 can be taken apart to separate support structures 100 and 108. A seal 114 is peripherally located on an interior surface of support structure 108. In one embodiment, the seal is an o-ring that comprises a compliant and compressible material. The seal 114 compresses and forms a seal against the interior surface of support structures 100 and 108 when attached together. Support structures 100 and 108 form a housing whereby the cavities or recesses within a boundary of seal 114 are isolated from an external environment. In one embodiment, a fastening element 120 illustrates an attaching mechanism. Fastening element 120 has a lip that couples to a corresponding fastening element on support structure 100. Fastening element 120 can have a canted surface to motivate coupling. Support structures 100 and 108 are fastened together when seal 114 is compressed sufficiently that the fastening elements interlock together. Support structures 100 and 108 are held together by fastening elements under force or pressure provided by seal 114 or other device/method such as a spring. Not shown are similar fastening elements that may be placed in different locations to secure support structures 100 and 108 equally around the perimeter if required.

In one embodiment, support structure 100 comprises material commonly used for passive inserts. For example, ultra high molecular weight polyethylene can be used. The material can be molded, formed, or machined to provide the appropriate support and articular surface thickness for a final insert. Alternatively, support structures 100 and 108 can be made of metal, plastic, or polymer material of sufficient strength for a trial application. In an intra-operative example, support structures 100 and 108 can be formed of polycarbonate. It should be noted that the long-term wear of the articular surfaces is a lesser issue for the short duration of the joint installation. The joint moves similarly to a final insert when moved throughout the range of motion with a polycarbonate articular surface. Support structure 100 can be a formed as a composite where a bearing material such as ultra high molecular weight polyethylene is part of the composite material that allows the sensing system to be used both intra-operatively and as a final insert.

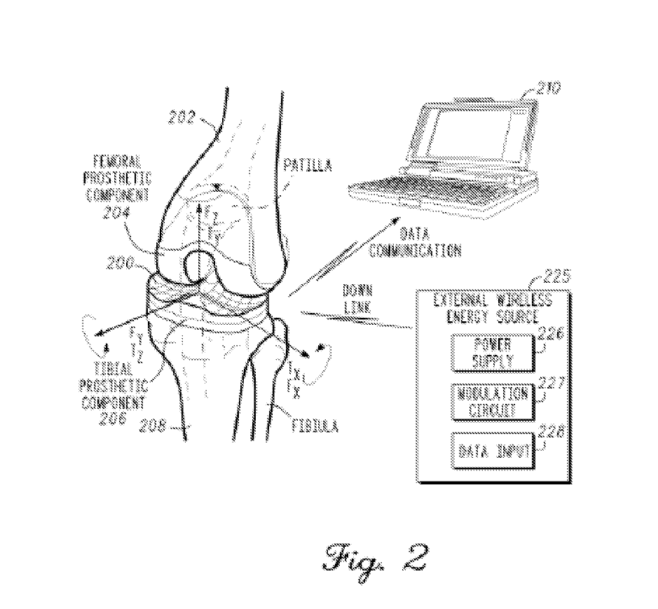

FIG. 2 illustrates an application of an insert sensing device 200 in accordance with an example embodiment. In general, one or more natural components of the muscular-skeletal system are replaced when joint functionality substantially reduces a patient quality of life. A joint replacement is a common procedure in later life because of wear, damage, or pain. Joint reconstruction can reduce pain while increasing patient mobility thereby allowing a return to normal activity. In this example, the insert sensing device 200 can intra-operatively assess a load on the prosthetic knee components (implant) and collect load data for real-time viewing of the load over various applied loads and angles of flexion and rotation. By way of an integrated antenna, a compact low-power energy source, and associated transceiver electronics, the insert sensing device 200 can transmit measured load data to a receiver for permitting visualization of the level and distribution of load at various points on the prosthetic components. This can aid the surgeon in making any adjustments needed to achieve optimal joint load and balance.

In general, an insert has at least one articular surface that allows articulation of the muscular-skeletal in conjunction with another prosthetic component. The insert is the wear component of a prosthetic joint and as used today is a passive component with no sensing or measurement capability. The insert is typically made of a solid block of polymer material that is resistant to wear, provides cushioning under loading, and is low friction. The block of polymer material is shaped to fit between other prosthetic components of the artificial joint. One such polymer material used for inserts is ultra-high molecular weight polyethylene.

A joint of the muscular-skeletal system provides movement of bones in relation to one another that can comprise angular and rotational motion. The joint can be subjected to loading and torque throughout the range of motion. A natural joint typically comprises a distal and proximal end of two bones coupled by one or more articular surfaces with a low friction, flexible connective tissue such as cartilage. The natural joint also generates a natural lubricant that works in conjunction with the cartilage to aid in ease of movement. Muscle, tendon, and ligaments hold the joint together and provide motivation for movement. Insert sensing device 200 mimics the natural structure between the bones of the joint. Insert sensing device 200 has at least one articular surface that allows articulation of the muscular-skeletal system. A knee joint is disclosed for illustrative purposes but insert sensing device 200 is applicable to other joints of the muscular-skeletal system. For example, the hip, spine, and shoulder have similar structures comprising two or more bones that move in relation to one another. In general, insert sensing device 200 provides parameter measurement over a range of motion of the muscular-skeletal system.

In the illustrated example, the insert sensing device 200 is a knee insert. The knee insert device 200 has two major surfaces. A first major surface of insert sensing device 200 contacts a distal end of femur 202. More specifically, insert sensing device 200 has an articular surface that allows a surface of femoral prosthetic component 204 to rotate allowing change in position of the tibia 108 in relation to femur 102. A second major surface of insert sensing device 200 contacts a tibial prosthetic component 206. The muscle, tendons, and ligaments hold the joint together and place a compressive force on the first and second major surfaces of device 200 when installed correctly. The compressive force allows free movement of the joint while retaining the joint in place over the range of motion and under various loadings. Measurement by insert sensing device 200 allows precise measurement and adjustment such that a force, pressure, or load is set during the trial phase of implantation. The final insert when installed will see a similar force, pressure, or load because the final insert and the trial insert are dimensionally substantially equal. It should be noted that device 200 is designed to be used in the normal flow of an orthopedic surgical procedure without special procedures, equipment, or components. As mentioned previously, device 200 has substantially equal dimensions as a passive final insert of the joint. Dimensional equivalence allows the insert sensing device 200 to be used both for trial and as a final insert having measurement capability.

The insert sensing device 200 and the receiver station 210 forms a communication system for conveying data via secure wireless transmission within a broadcasting range over short distances on the order of a few meters to protect against any form of unauthorized or accidental query. In one embodiment, the transmission range is five meters or less which is approximately a dimension of an operating room. In practice, it can be a shorter distance 1-2 meters to transmit to a display outside the sterile field of the operating room. The transmit distance will be even shorter when device 200 is used in a prosthetic implanted component. Transmission occurs through the skin of the patient and is likely limited to less than 0.5 meters. A combination of cyclic redundancy checks and a high repetition rate of transmission during data capture permits discarding of corrupted data without materially affecting display of data

In the illustration, a surgical procedure is performed to place the femoral prosthetic component 204 onto a prepared distal end of the femur 202. Similarly, a tibial prosthetic component 206 is placed to a prepared proximal end of the tibia 208. The tibial prosthetic component 206 often is a tray or plate affixed to a planarized proximal end of the tibia 208. The insert sensing device 200 is a third prosthetic component that is placed between the plate of the tibial prosthetic component 206 and the femoral prosthetic component 204. The three prosthetic components enable the prostheses to emulate the functioning of a natural knee joint. In one embodiment, insert sensing device 200 is used during surgery and replaced with a final insert after quantitative measurements are taken to ensure optimal fit, balance, and loading of the prosthesis.

As mentioned previously, insert sensing device 200 is dimensionally equivalent to a final insert from an operational perspective. The device 200 fits similarly within the joint as the final insert but is substantially equivalent from an operational perspective. Operational equivalency ensures that parameter measurements made by insert sensing device 200 will translate to the final insert or be equivalent to what is applied to the final insert by the muscular-skeletal system. In at least one embodiment, insert sensing device 200 has substantially equal dimensions to the final insert. There can be differences that are non-essential from a measurement perspective between device 200 and the final insert. The substantial equal dimensions ensure that the final insert when placed in the reconstructed joint will have similar loading and balance as that measured by insert sensing device 200 during the trial phase of the surgery. The substantially equal dimensions also allow fine adjustment such as soft tissue tensioning by providing access to the joint region. Moreover, passive trial inserts are commonly used during surgery to determine the appropriate final insert. Thus, the procedure remains the same and familiar to the surgeon. It can measure loads at various points (or locations) on the femoral prosthetic component 204 and transmit the measured data to a receiving station 210 by way of an integrated antenna. The receiving station 210 can include data processing, storage, or display, or combination thereof and provide real time graphical representation of the level and distribution of the load.

As one example, the insert sensing device 200 can measure forces (Fx, Fy, and Fz) with corresponding locations and torques (e.g. Tx, Ty, and Tz) on the femoral prosthetic component 204 and the tibial prosthetic component 206. It can then transmit this data to the receiving station 210 to provide real-time visualization for assisting the surgeon in identifying any adjustments needed to achieve optimal joint balancing.

In a further example, an external wireless energy source 225 can be placed in proximity to the insert sensing device 200 to initiate a wireless power recharging operation. As an example, the external wireless energy source 225 generates energy transmissions that are wirelessly directed to the insert sensing device 200 and received as energy waves via resonant inductive coupling. The external wireless energy source 225 can modulate a power signal generating the energy transmissions to convey downlink data that is then demodulated from the energy waves at the medical sensing device 200. As described above, the insert sensing device 200 is an insert suitable for use as a trial or a permanent knee joint replacement surgery. The external wireless energy source 225 can be used to power the insert sensing device 200 during the surgical procedure or thereafter when the surgery is complete and the device 200 is implanted for long-term use. The method can also be used to provide power and communication where the insert sensing device 200 is in a final insert that is part of the final prosthesis implanted in the patient. The integration of the patient's own load during walking or movement can be coupled by converting this kinetic energy into energy to power the system. This is referred herein as energy harvesting.

In one system embodiment, the insert sensing device 200 transmits measured parameter data to a receiver 210 via one-way data communication over the up-link channel for permitting visualization of the level and distribution of the parameter at various points on the prosthetic components. This, combined with cyclic redundancy check error checking, provides high security and protection against any form of unauthorized or accidental interference with a minimum of added circuitry and components. This can aid the surgeon in making any adjustments needed to optimize the installation. In addition to transmitting one-way data communications over the up-link channel to the receiver station 210, the insert sensing device 200 can receive downlink data from the external wireless energy source 225 during the wireless power recharging operation. The downlink data can include component information, such as a serial number, or control information, for controlling operation of the insert sensing device 200. This data can then be uploaded to the receiving system 210 upon request via the one-way up-link channel, in effect providing two-way data communications over separate channels.

As shown, the wireless energy source 225 can include a power supply 226, a modulation circuit 227, and a data input 228. The power supply 226 can be a battery, a charging device, a capacitor, a power connection, or other energy source for generating wireless power signals to power the insert sensing device 200. The external wireless energy source can transmit energy in the form of, but not limited to, electromagnetic induction, or other electromagnetic or ultrasound emissions. In at least one example embodiment, the wireless energy source 225 includes a coil to electromagnetically couple with an induction coil in sensing device 200 when placed in close proximity. Alternatively, energy harvesting can be used to charge and power insert sensing device 200. The data input 228 can be a user interface component (e.g., keyboard, keypad, or touch screen) that receives input information (e.g., serial number, control codes) to be downloaded to the insert sensing device 200. The data input 228 can also be an interface or port to receive the input information from another data source, such as from a computer via a wired or wireless coupling (e.g., USB, IEEE802.16, etc.). The modulation circuitry 227 can modulate the input information onto the power signals generated by the power supply 226.

Separating uplink and downlink telemetry eliminates the need for transmit-receive circuitry within the insert sensing device 200. Two unidirectional telemetry channels operating on different frequencies or with different forms of energy enables simultaneous up and downlink telemetry. Modulating energy emissions from the external wireless energy source 225 as a carrier for instructions achieves these benefits with a minimum of additional circuitry by leveraging existing circuitry, antenna, induction loop, or piezoelectric components on the insert sensing device 200. The frequencies of operation of the up and downlink telemetry channels can also be selected and optimized to interface with other devices, instruments, or equipment as needed. Separating uplink and downlink telemetry also enables addition of downlink telemetry without altering or upgrading existing chip-set telemetry for the one-way transmit. That is, existing chip-set telemetry can be used for encoding and packaging data and error checking without modification, yet remain communicatively coupled to the separate wireless power down-link telemetry operation for download operations herein contemplated. Alternatively, insert sensing device 200 can be fitted with a standardized wireless transmit and receive circuitry such as Bluetooth, Zigbee, UWB, or other known wireless systems to communicate with receiver station 210.

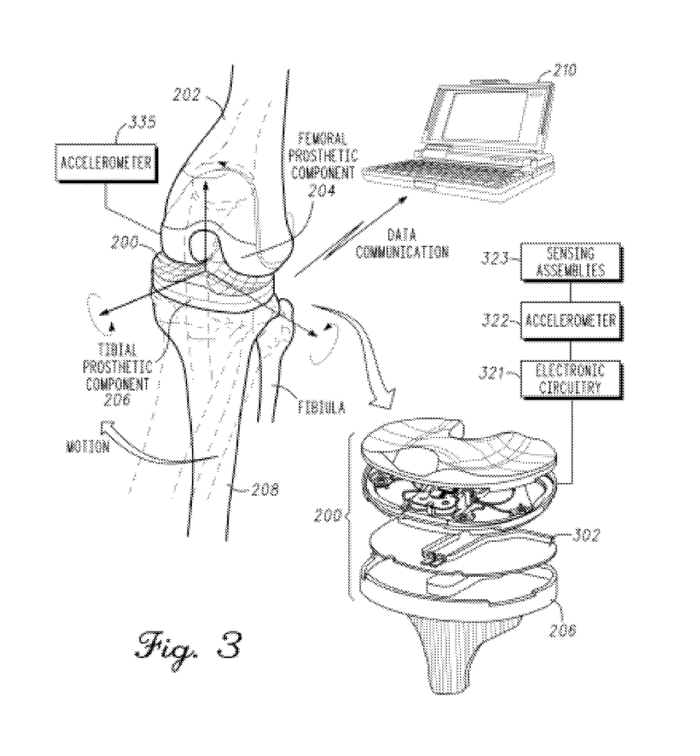

FIG. 3 illustrates an insert sensing device 200 placed in a joint of the muscular-skeletal system for measuring a parameter in accordance with an example embodiment. In particular, insert sensing device 200 is placed in contact between a femur 202 and a tibia 208 for measuring a parameter. In the example, a force, pressure, or load is being measured. The device 200 in this example can intra-operatively assess joint loading of installed prosthetic components during the surgical procedure. The insert sensing device 200 can measure the magnitude and distribution of load at various points on the prosthetic component while transmitting the measured load data by way of wireless data communication to a receiver station 210 for real-time visualization. This can aid the surgeon in making any adjustments needed to achieve optimal joint loading and balance.

A proximal end of tibia 208 is prepared to receive tibial prosthetic component 206. Tibial prosthetic component 206 is a support structure that is fastened to the proximal end of the tibia and is usually made of a metal or metal alloy. The tibial prosthetic component 206 also retains the insert in a fixed position with respect to tibia 208. Similarly, a distal end of femur 202 is prepared to receive femoral prosthetic component 204. The femoral prosthetic component 204 is generally shaped to have an outer condylar articulating surface. The preparation of femur 202 and tibia 208 is aligned to the mechanical axis of the leg. The upper major surface of insert sensing device 200 provides a concave or flat surface against which the outer condylar articulating surface of the femoral prosthetic component 204 rides relative to the tibial prosthetic component 206 allowing movement of tibia 208 in relation to femur 202. Conversely, the lower major surface of insert sensing device 200 is non-articulating and couples to the major exposed surface of the tibial prosthetic component 206. The height of insert sensing device 200 can be adjusted during surgery by adding one or more shims of different height to affect the loading thereto. In one embodiment, the load-bearing surface of insert sensing device 200 does not interface with a tibial prosthetic component 206. Shim 302 can be required as part of insert sensing device 200. Shim 302 can be designed to align with and be retained for a specific tibial prosthetic component. This is beneficial in providing flexibility in supporting many different types of prosthetic component families with a single measurement system. Shim 302 is a passive low cost component that can be provided in many shapes and sizes. Alternatively, insert sensing device 200 can be shaped for a specific tibial prosthetic component such that device 200 can only be mated to the tibial prosthetic component or a family of prosthetic components. Shim 302 attaches by one or more fasteners to the lower major surface of insert sensing device 200. Adding shims increases a height of device 200 thereby raising the compressive force applied by the joint to the major surfaces of the device 200. Shim 302 when attached to device 200 has an exposed major surface for interfacing with tibal prosthetic component 206.

The insert sensing device 200 is used to measure, adjust, and test the reconstructed joint prior to installing the final insert. As mentioned previously, the insert sensing device 200 is inserted between the femur 202 and tibia 208. In a total knee reconstruction a condyle surface of femoral component 204 contacts a corresponding articular surface on device 200. The major surface of device 200 approximates or is identical to a surface of a final insert. In particular, the contact area of the femoral component 204 to the articular surface of device 200 is substantially equal to or can be correlated to the contact area between the femoral component 204 and the final insert. Tibial prosthetic component 206 has an exposed major surface that receives and retains insert sensing device 200 during a measurement process. In one embodiment, device 200 is provided having different sizes and shapes to fit different tibial prosthetic components. It should be noted that insert sensing device 200 is coupled to and can provide measurement data in conjunction with other implanted prosthetic components. Thus, in one embodiment, device 200 is used to generate parameter measurements as a trial insert with other final prosthetic components. This ensures that the final insert, when inserted, will see loading and balance similar to that applied to the trial insert.

In general, prosthetic components are made in different sizes to accommodate anatomical differences over a wide population range. Similarly, insert sensing device is designed for different prosthetic sizes and shapes. Internally, each sensing device will have similar electronics and sensors. The mechanical layout and structure will also be similar between different sized units. The main variable during trial insertion is the insert height. The height or thickness of insert sensing device 200 is adjusted by one or more shims 302. The surgeon selects shim 302 based on the gap between the femur and tibial cuts after preparation of the bone surfaces. The insert sensing device 200 of a predetermined height is then inserted in the knee joint to interact with the final femoral prosthetic component 204 and tibial prosthetic component 206. The surgeon may try changing the height or thickness using different shims before making a final decision on the appropriate dimensions of the final insert. Each trial by the surgeon can include modifications to the joint and tissue. In one embodiment, insert sensing device 200 selected by the surgeon has substantial equal dimensions to the final insert used. The insert sensing device 200 allows standardization for a prosthetic platform while providing familiarity of use and installation. Thus, the insert sensing module 200 can easily migrate from a trial insert to a final insert that allows long-term monitoring of the joint.

In one embodiment, the insert sensing device 200 is used to measure, a force, pressure or load in one or more compartments of the knee. Data from device 200 is transmitted to a receiving station 210 via wired or wireless communications. The surgeon can view the transmitted information on a display. The effect of an adjustment by the surgeon is viewed in real-time with quantitative measurement feedback from device 200. The surgeon uses the trial insert to determine an appropriate thickness for the final insert that yields an optimal load and balance. The absolute loading is monitored over the entire range of motion. The magnitude of the loading in each compartment of the knee is kept within a predetermined range. The insert sensing module 200 is removed and modified with a shim if the absolute loading is found to be below the predetermined range. The modified insert sensing module 200 having an increased height due to shim 302 is then re-inserted into the knee joint. Muscular-skeletal adjustments and shim adjustments are made until the loading in each compartment is within the predetermined range.

Once the measurements indicate that the measured loading is within the predetermined range, soft tissue tensioning or bony cut refinements can be used to adjust the absolute loading. Similarly, the knee balance is adjusted by soft tissue tensioning such that the measured differential loading between compartments falls within a predetermined range for a total knee reconstruction. The balance predetermined range corresponds to the differential between the loads measured in each compartment. It should be noted that the balance does not have to be equal. Optimal balance can be a non-equal differential loading between the medial and lateral compartments. Furthermore, the position or location of the applied force, pressure, or load occurs on the articular surfaces can also be measured by insert sensing device 200 allowing the surgeon to adjust contact location over the range of motion. In particular, it is not desirable for the loading to be towards the outer edge of the articular surface. Device 200 identifies where and at what position the edge loading occurs such that an adjustment can be made. Thus, the surgeon uses the quantitative data from insert sensing device 200 to select a height of the final insert and to make adjustments on the absolute loading, balance, and position. The adjustments can be made with the joint in one or more positions. In one embodiment, measurements are taken in extension and flexion. In one embodiment, insert sensing device 200 is a disposable device that is disposed of as hazardous waste after surgery. Alternatively, the insert sensing device 200 and shim 302 can be sterilized and packaged for reuse.

In one embodiment, a passive final insert is fitted between femoral prosthetic component 204 and tibial prosthetic component 206 based on quantitative measurement data. The final insert has at least one articular surface that couples to femoral component 204 allowing the leg a natural range of motion. The region between the two articular surfaces of a total knee reconstruction insert is a lightly loaded or un-loaded region of the insert. As mentioned above, the final insert has a wear surface that is typically made of a low friction polymer material. Ideally, the prosthesis has a loading, alignment, and balance that mimic a natural leg. It should be noted that insert sensing device 200 can be placed as a final insert and operated similarly as disclosed herein. The wear surface can comprise one or more layers of low friction polymer material can be bonded or attached to a housing of device 200 to form the articular surfaces. Alternatively, the upper and lower support structures that form a housing of device 200 can be molded or machined from the low friction polymer material.

In a first embodiment, device 200 is a low cost disposable system that reduces capital costs, operating costs, facilitates rapid adoption of quantitative measurement, and initiates evidentiary based orthopedic medicine. In a second embodiment, a methodology can be put in place to clean and sterilize device 200 for reuse. In a third embodiment, device 200 can be incorporated in a tool instead of being a component of the replacement joint. The tool can be disposable or be cleaned and sterilized for reuse. In a fourth embodiment, device 200 can be a permanent component of the replacement joint. Device 200 can be used to provide both short term and long term post-operative data on the implanted joint. In a fifth embodiment, device 200 can be coupled to the muscular-skeletal system in a non-joint application for parameter measurements. In all of the embodiments, receiving station 210 can include data processing, storage, or display, or combination thereof and provide real time graphical representation of the level and distribution of the load. Receiving station 210 can record and provide accounting information of device 200 to an appropriate authority.

The insert sensing device 200, in one embodiment, comprises electronic circuitry 321, an accelerometer 322, and sensing assemblies 323 which can include a gyroscope. This permits the insert sensing device 200 to assess a total load on the prosthetic components as the joint is taken through the range of motion. The system accounts for forces due to gravity and motion. The accelerometer 322 of device 200 measures acceleration. Acceleration can occur when the sensing device 200 is moved or put in motion. Accelerometer 322 senses orientation, vibration, and impact. In another embodiment, the femoral component 204 can similarly include an accelerometer 335 and a gyroscope, which by way of a communication interface communicates to the insert sensing device 200, thereby providing reference position and acceleration data to determine an exact angular relationship between the femur 202 and tibia 208. In one embodiment, sensing assemblies 323 can reveal changes in length or compression of the energy propagating structure or structures by way of the energy transducer or transducers. Together the electronic circuitry 321, accelerometer 322, accelerometer 335, and sensing assemblies 323 measure force or pressure external to the load sensing platform 321 or displacement produced by contact with the prosthetic components.

Incorporating data from the accelerometer 322 with data from the electronic circuitry 321 and sensing assemblies 323 assures accurate measurement of the applied load, force, pressure, or displacement by enabling computation of adjustments to offset this external motion. This capability can be required in situations wherein the body, instrument, appliance, vehicle, equipment, or other physical system, is itself operating or moving during sensing of load, pressure, or displacement. This capability can also be required in situations wherein the body, instrument, appliance, vehicle, equipment, or other physical system, is causing the portion of the body, instrument, appliance, vehicle, equipment, or other physical system being measured to be in motion during sensing of load, pressure, or displacement.

The accelerometer 322 with or without the gyroscope can operate singly or as an integrated unit with the electronic circuitry 321 and the sensing assemblies 323. Integrating one or more accelerometers 322 within the sensing assemblages 323 to determine position, attitude, movement, or acceleration of sensing assemblages 323 enables augmentation of presentation of data to accurately identify, but not limited to, orientation or spatial distribution of load, force, pressure, displacement, density, or viscosity, or localized temperature by controlling the load and position sensing assemblages to measure the parameter or parameters of interest relative to specific orientation, alignment, direction, or position as well as movement, rotation, or acceleration along any axis or combination of axes. Measurement of the parameter or parameters of interest may also be made relative to the earth surface and thus enable computation and presentation of spatial distributions of the measured parameter or parameters relative to this frame of reference.

In one embodiment, the accelerometer 322 includes direct current (DC) sensitivity to measure static gravitational pull with load and position sensing assemblages to enable capture of, but not limited to, distributions of load, force, pressure, displacement, movement, rotation, or acceleration by controlling the sensing assemblages to measure the parameter or parameters of interest relative to orientations with respect to the earths surface or center and thus enable computation and presentation of spatial distributions of the measured parameter or parameters relative to this frame of reference.

Embodiments of device 200 are broadly directed to measurement of physical parameters, and more particularly, to evaluating changes in the transit time of a pulsed energy wave propagating through a medium. In-situ measurements during orthopedic joint implant surgery would be of substantial benefit to verify an implant is in balance and under appropriate loading or tension. In one embodiment, the instrument is similar to and operates familiarly with other instruments currently used by surgeons. This will increase acceptance and reduce the adoption cycle for a new technology. The measurements will allow the surgeon to ensure that the implanted components are installed within predetermined ranges that maximize the working life of the joint prosthesis and reduce costly revisions. Providing quantitative measurement and assessment of the procedure using real-time data will produce results that are more consistent. A further issue is that there is little or no implant data generated from the implant surgery, post-operatively, and long term. Device 200 can provide implant status data to the orthopedic manufacturers and surgeons. Moreover, data generated by direct measurement of the implanted joint itself would greatly improve the knowledge of implanted joint operation and joint wear thereby leading to improved design and materials.

As mentioned previously, device 200 can be used for other joint surgeries; it is not limited to knee replacement implant or implants. Moreover, device 200 is not limited to trial measurements. Device 200 can be incorporated into the final joint system to provide data post-operatively to determine if the implanted joint is functioning correctly. Early determination of a problem using device 200 can reduce catastrophic failure of the joint by bringing awareness to a problem that the patient cannot detect. The problem can often be rectified with a minimal invasive procedure at lower cost and stress to the patient. Similarly, longer term monitoring of the joint can determine wear or misalignment that if detected early can be adjusted for optimal life or replacement of a wear surface with minimal surgery thereby extending the life of the implant. In general, device 200 can be shaped such that it can be placed or engaged or affixed to or within load articular surfaces used in many orthopedic applications related to the musculoskeletal system, joints, and tools associated therewith. Device 200 can provide information on a combination of one or more performance parameters of interest such as wear, stress, kinematics, kinetics, fixation strength, ligament balance, anatomical fit and balance.

FIG. 4 illustrates an adjustable height insert sensing device 400 in accordance with an example embodiment. Insert sensing device 400 comprises a housing 402. Housing 402 has at least one articular surface and a load bearing surface allowing articulation of the muscular-skeletal system. Housing 402 is a self-contained measurement system that includes a power source, electronic circuitry, and sensors for measuring a parameter of the muscular-skeletal system. For illustrative purposes, insert sensing device 400 is a knee insert for total knee reconstruction. Insert sensing device 400 as shown has a major surface 406 that includes two articular surfaces corresponding to each compartment of the knee. In one embodiment, each articular surface has a concave shape for receiving a prosthetic femoral condyle. A major surface 408 of housing 402 relates to a tibial prosthetic component. In general, the tibial prosthetic component when installed has an exposed tray or surface for receiving and retaining insert sensing device 400. In one embodiment, major surface 408 interfaces with a major surface of the tibial tray of the tibial prosthetic component. The interface between major surface 408 and the planar region of the tibial tray distributes the load over the region. Thus, the major surface 408 is a load-bearing surface. The major surface 408 has a predetermined shape that aligns with and is retained in a fixed relational position to the tibial tray. Typically, the contact area between the tibial tray and device 400 is greater than the contact area of the prosthetic femoral condyles to the articular surfaces. The loading on surface 408 is reduced through distribution of the force over a larger area than occurs on the articular surfaces.

The minimum height of insert sensing device 400 comprises housing 402 without shim 404. The insert sensing device further comprises a plurality of shims each having a different height. In a further embodiment, the shims can be stacked to form different heights. Shim 404 is a passive device for modifying the height of insert sensing device 400. In one embodiment insert sensing device 400 requires at least one shim to interface with a corresponding prosthetic component. The shim is shaped as the interface device to the prosthetic component. Thus, the insert sensing device 400 can be used with a variety of different prosthetic component system. The surgeon prepares the knee joint such that a femoral prosthetic component is attached to the distal end of the femur and the tibial prosthetic component is attached to the proximal end of the tibia. The initial bone cuts and preparation are made by the surgeon to provide a sufficient gap to accommodate insert sensing device 400 with the tibial and femoral prosthetic components attached. In one embodiment, the gap left between the tibial and femoral prosthetic components is greater than or equal to the height or thickness of insert sensing device 400 comprising only housing 402.

The insert sensing device 400 is placed between the femoral and tibial prosthetic components. The tibial prosthetic component typically has one or more features to retain an insert in place after insertion in the joint. The muscle, ligaments, and tendons stretch to accommodate placement of the insert in the joint and retract once the prosthetic component is seated between the tibia and femur. The muscle, ligaments, and tendons apply a compressive force on the insert sensing device 400. Typically, the gap is designed by the surgeon to be greater than the height or thickness of housing 402 such that a shim is required to generate a retaining compressive force on the major surfaces of insert sensing device 400 after insertion. Shims of different heights or thicknesses, such as shim 404, are used to determine an appropriate thickness for the final insert. In one embodiment, the height or thickness of insert sensing device 400 is selected to measure higher than optimal when inserted. Soft tissue tensioning is then used to adjust absolute magnitude in each compartment and adjust balance between compartments.

The shim 404 is attachable to the major surface 408 of housing 402. Shim 404 has a major surface 410 and a major surface 412. Shim 404 has a predetermined height or thickness. The predetermined height or thickness of shim 404 is the distance between major surfaces 410 and 412. Major surface 410 interfaces with major surface 408 of housing 402. In one embodiment, housing 402 has slots 414 and tab 416. Shim 404 has tabs (not shown) and a slot 418. The major surface 410 is positioned to interface with major surface 408 of housing 402. The major surface 410 slideably engages with the major surface 408 of housing 402 until the tabs are inserted into slots 414 and tab 416 locks into slot 418 thereby retaining shim 404 onto housing 402. A force is applied to shim 404 to engage tab 416 to slot 418 that retains the shim 404 to housing 402. The retaining force can be released when tab 416 is depressed to disengage tab 416 from slot 418 thereby allowing separation of shim 404 from housing 402. The shim 404 coupled to housing 402 has surface 412 exposed. Surface 402 has a footprint substantially dimensionally equal to the footprint of major surface 408 of the housing 402 to engage with a tibial prosthetic component. The height of insert sensing device 400 is the combined height or thickness of housing 402 and shim 404. Shim 404 can be separated from housing 402 by depressing tab 416 and sliding shim 404 from housing 402. The use of shims allows rapid changing of the height and angles of insert sensing device 400. Moreover, the feedback provided to the surgeon using the trial insert is both subjective through movement of the joint and quantitative from the measurement sensors in housing 402. Finally, the device 400 allows fine-tuning of the loading and balance within suggested predetermined ranges based on quantitative data. The predetermined ranges can be based on collected data from a large number of patients using device 400 both intra-operatively and long-term.

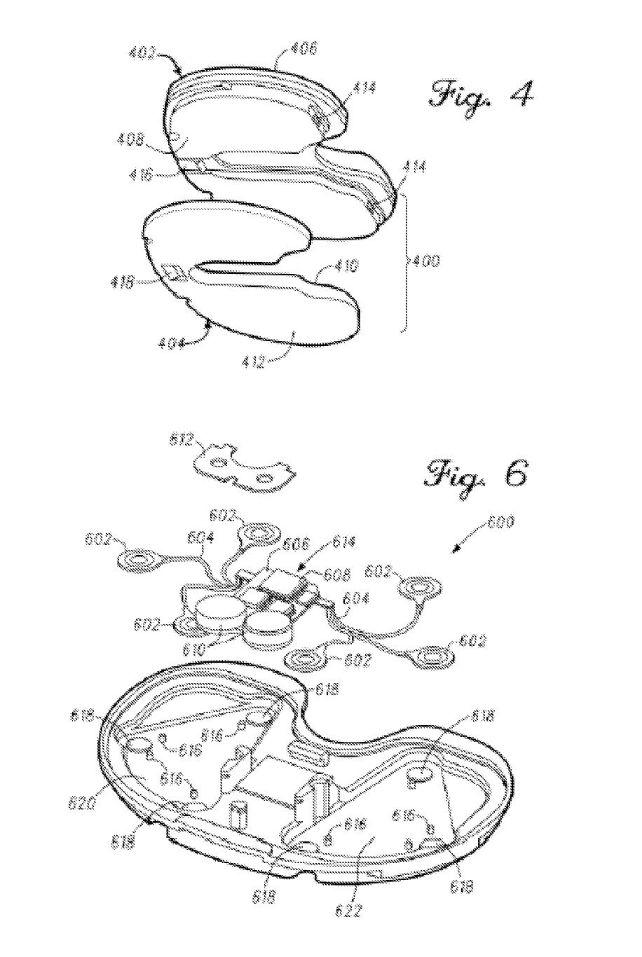

FIG. 5 illustrates an insert sensing device 500 comprising a housing 512 and a plurality of shims 514 in accordance with an example embodiment. The insert sensing device 500 includes at least one sensor, electronic circuitry, and a power source for measuring a parameter of the muscular-skeletal system. In one embodiment, sensors underlie articular surfaces 516 and 518 for measuring an applied force, pressure, or load. Articular surfaces 516 and 518 are articular surfaces of a knee joint. The sensors measure the load magnitude and the location where the load is applied to articular surfaces 516.

A tibial prosthetic component 506 interfaces with insert sensing device 500. Tibial prosthetic component 506 includes a major surface 506 and a stem 510. After the surgeon prepares the proximal end of a tibia, the stem 510 of prosthetic component 506 is inserted into the medullary cavity of the bone. The stem 510 supports, retains, and stabilizes tibial prosthetic component 506 in the tibia. A tibial tray that includes major surface 508 is exposed for receiving an insert. As shown, the tibial tray has a sidewall 520 extending around the perimeter of the major surface 508. The major surface 508 supports each compartment of the knee in conjunction with the tibia.

The housing 512 by itself or in combination with one of shims 514 are inserted and removed from the tibial tray during the reconstructive knee surgery until a final insert height or thickness is determined. The shape of bottom surface of housing 512 or shims 514 is similar to the tibial tray. The bottom surface of housing 512 or shims 514 contacts major surface 508 when installed. In the illustration, the sidewall 520 and the compressive force applied by the joint retains insert sensing device 500 in the tibial tray throughout the range of motion of the joint.

A shim is shown having a raised sidewall 502 with tabs 504 and a slot 522. Although a single shim has the identified features, each of shims 514 has an identical sidewall, tabs, and slot. As disclosed hereinabove, shims 514 slideably attach to housing 512 to increase the height or thickness of insert sensing device 500. Although not shown, the sidewall of housing 512 can be recessed to accommodate the thickness of raised sidewall 502. The recess aligns the sidewall 502 to the sidewall of the housing 512. In one embodiment, cavities are formed in shims 514 to reduce the amount of material used in the manufacture of the component. The remaining major surface area of shims 514 is sufficient to support and distribute the loading applied to insert sensing device 500. The cavities also enhance or maintain the structural integrity of shims 514. In one embodiment, each shim and housing combination corresponds to an available final insert thickness. The appropriate device size is determined by loading and balance measurements. Fine adjustments such as soft tissue tensioning are made with the selected insert sensing device 500. In one embodiment, insert sensing device 500 is then removed, disposed of, and a final insert inserted into the joint having the same height or thickness as the trial insert. The load and balance on the final insert is similar to that of the previously removed insert sensing device 500. Moreover, insert sensing device 500 is substantially dimensionally equal to the final insert to minimize operational differences between the measurements and subjective feel of device 500 and the final insert in the muscular-skeletal system. As mentioned previously, the insert sensing device 500 can be the final insert. Although shims 514 are shown comprising 5 shims of different height, there can be more or less shims made for the measurement system depending on the change in loading between shims required for the application.

A method of adjusting the height of an insert sensing device is supported by the embodiment disclosed herein. The steps disclosed herein can be performed in any order or combination. In the method, a parameter of the muscular-skeletal system is measured. In a first step, the insert is provided having an articulating surface and load-bearing surface. The articular surface of the insert allows movement of the muscular-skeletal system. The insert is a housing for the self-contained measurement system. In a second step, a shim of a predetermined height is coupled to the load bearing surface. Bones of the muscular-skeletal system are prepared and receive one or more prosthetic components. In one embodiment, the height or thickness of the insert sensing device including the shim corresponds to the gap between the prosthetic components coupled to the bones of the joint. In a third step, the insert with shim is inserted in the joint of the muscular-skeletal system. The measuring system within the insert sensing device is then enabled to measure one or more parameters. In the example, the measured parameter is a force, pressure, distance, or load applied by the muscular-skeletal system to the one or more articular surfaces of the insert sensing device. The quantitative measurements are used in conjunction with subjective measurements made by the surgeon as the joint is moved through a range of motion.

In one example, the qualitative and quantitative measurements with the device insert indicate that insufficient loading is being applied to the articular surface of the insert sensing device. An insert having an increased height is required to produce a loading measurement within a known optimal range. The insert sensing device is removed from the joint. In a fifth step, the shim is removed from the insert. In a sixth step, the shim is disengaged by releasing a force that retains the shim to the load-bearing surface of the insert. In the illustration, a tab and an opening respectively on the insert and shim are coupled together by retaining force. Pushing the tab inward disengages the tab from the opening thereby removing the retaining force. The shim can then be removed from the insert.

In a seventh step, a shim is slideably attached to the insert. Using the example disclosed herein above, the added shim is thicker or has a greater height than the previously removed shim to increase the overall height of the insert once attached. A major surface of the shim is placed in contact with the load-bearing surface of the insert. The surfaces of the shim and insert slide such that the major surface of the shim overlies the load-bearing surface of the insert and are coupled together. The exposed major surface of the shim is substantially dimensionally equal to the load-bearing surface of the insert. In an eighth step, the insert and shim are aligned in a specific orientation before being slideably engaged. In particular, one or more tabs on a sidewall of the shim align to openings in the sidewall of the insert. The tabs further aid in retaining the shim to the insert. The surfaces of the shim and insert slide against each other and are oriented such that the tabs are inserted into the corresponding openings. In a ninth step, the shim is retained under force to the insert. A retaining feature comprises a tab and slot that engage when the tab is aligned to the slot such that a surface of the tab interfaces with a surface in the slot. A force is applied between the insert and shim to align the tab and slot. Once engaged, the force retains the shim to the insert. As mentioned previously, the tab can be moved to disengage from the slot thereby removing the retaining or holding force thereby allowing removal of the shim from the insert. The insert with increased height can be reinserted in the joint. The surgeon performs an iterative process of qualitative and quantitative measurements using inserts of different heights until the data yields results within a known operational range that ensures optimal joint performance and longevity. This process can require that the insert be removed and the shim replaced multiple times. In one embodiment, the final insert having substantially equal height or thickness is then selected after the intra-operative shimming procedure is performed. The final insert typically is not shimmed but is provided having the selected height or thickness. A passive final insert will comprise a shaped block of polymer material. The final insert can include a measurement system similar to that used in the intra-operative procedure.

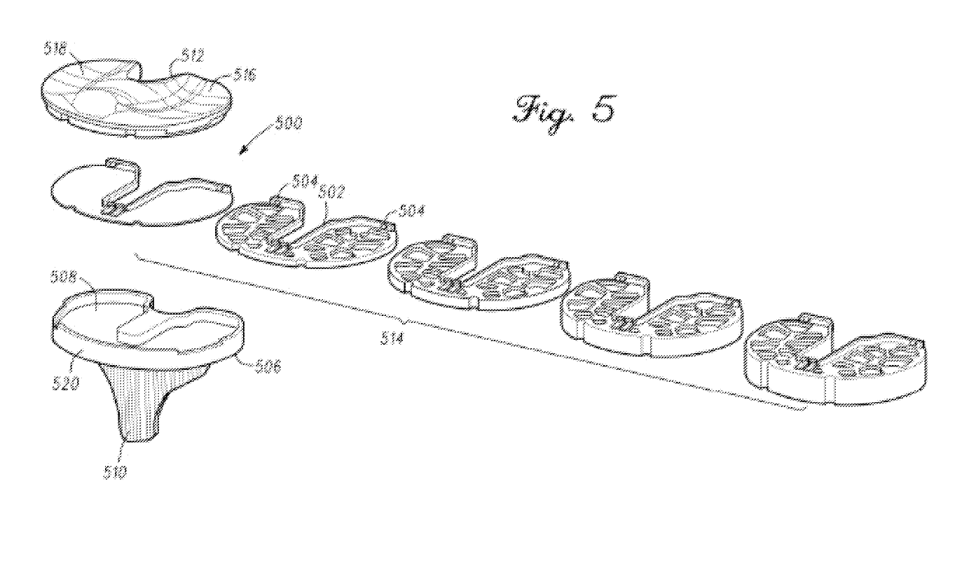

FIG. 6 illustrates a lower support structure 600 of an insert sensing device in accordance with an example embodiment. An upper support structure (not shown) has at least one bearing or articular surface to allow movement of the muscular-skeletal system. The upper support structure fastens to the lower support structure 600 to form a sealed enclosure. The sealed enclosure is an active component of an insert for parameter measurement to aid in prosthetic installation, muscular-skeletal parameter measurement or long-term monitoring of a reconstructed joint. The entire measurement system is self-contained within the upper and lower support structure. As shown, the measurement system fits within the dimensions of a prosthetic component. For illustrative purposes, the upper and the lower support structure 600 houses multiple sensors for measuring the magnitude and position of loading applied to each compartment of a knee insert.

The active system of the insert comprises sensors 602, interconnect 604, one or more printed circuit boards 606, electronic circuitry 614, a power source 610, and a power source retainer 612. The electronic circuitry 614 is mounted on printed circuit board 606. The electronic circuitry 614 comprises power management circuitry, measurement circuitry, parameter conversion circuitry, and transmit/receive circuitry. In one embodiment, an application specific integrated circuit (ASIC) 608 for muscular-skeletal parameter sensing is utilized. The ASIC reduces the number of components that mount to printed circuit board 606. The integration of circuitry onto an ASIC eliminates unneeded circuitry, adds functions specific to parameter measurement, reduces power consumption of the measurement system, and reduces the sensing system form factor to a size that fits within a prosthetic component.

The power source 610 powers electronic circuitry 614 and sensors 602. In one embodiment, the power source 610 comprises one or more batteries. As shown, two batteries are coupled to the printed circuit board 610. The power source retainer 612 retains the batteries in place as will be shown hereinbelow. In one embodiment, the system is disposed of once the batteries have been depleted such as an intra-operative measurement procedure. Alternatively, a rechargeable system can power electronic circuitry 614. The power source 610 can be a rechargeable battery, capacitor, or other temporary power source. The power source 610 can be electro-magnetically coupled to a remote source for receiving charge. The power source 610 and power management circuitry enables the system for parameter measurement after sufficient charge is stored. It should be noted that the power consumption reduction due to the ASIC enables the use of rechargeable methodologies such as the capacitor. The capacitor provides the further benefit of extended life and no chemicals when compared with batteries for a long-term implant application such as joint monitoring.

In the example, the measurement system measures the loading, balance, and load location on each knee compartment. Each knee compartment includes three sensors for load measurement. In one embodiment, each sensor is a piezo-resistive film sensor. The resistance of a piezo-resistive film changes with an applied pressure. A resistance, voltage, or current corresponding to the piezo-resistive film under load is measured. The measured resistance, voltage, or current is then correlated back to a pressure measurement. In a second embodiment, a transit time is correlated to the pressure measurement. An ultrasonic continuous wave or pulsed signal is propagated through a compressible waveguide. Loading on the insert compresses the compressible waveguide thereby changing the length of the waveguide. A change in length corresponds to a change in transit time. The transit time can be related to a frequency by holding the number of waves in the compressible waveguide to a fixed integer number during a measurement sequence. Thus, measuring the transit time or frequency allows the length of the waveguide to be precisely measured. The pressure can be calculated with knowledge of the length versus applied pressure relationship of the waveguide. Other sensor types can also be used such as strain gauge, mems, and mechanical sensors.

The three sensors underlie the bearing or articular surface of the upper support structure. The three sensors of each compartment are located at predetermined positions of lower support structure 600. Measurements from the three sensors are used to determine the location where the load is applied to the corresponding articular surface. The electronic circuitry 614 can take measurements sequentially or in parallel. The location and magnitude of the applied load is determined by analysis of the magnitudes from each of the three sensors of a compartment. The analysis includes a differential comparison of the measured loads. In general, the location of the applied load is closer to the sensor reading the highest load magnitude. Conversely, the applied load will be farthest from the sensor having the lowest load magnitude. The use of three sensors allows the applied load location to be determined utilizing knowledge of the predetermined sensor locations.

The lower support structure 600 has a cavity 620 and a cavity 622 each underlying an articular surface of the upper support structure. In one embodiment, cavities 620 and 622 are triangular in shape. Pad regions 618 are located at the vertex of triangular cavities 620 and 622. The pad regions 618 are raised regions above a bottom surface of cavities 620 and 622 having a predetermined area and location. As shown, pad regions 618 are cylindrical in shape forming a short column. A sensor is placed on each pad region such that the sensor area for measurement corresponds to the predetermined area of pad region 618. Retaining structures 616 are used to retain and precisely locate the sensors within cavities 620 and 622. For example, a piezo-resistive film sensor is placed on each pad region 618. The predetermined area of pad regions 618 is selected to distribute the load over sufficient area for reliable sensing, provide a measurable signal (e.g. voltage, current, resistance) over the loading range, and have the sensitivity for precise measurement. The predetermined area and location is sufficiently small to allow accurate identification of the load location based on the measurements of the three sensors.

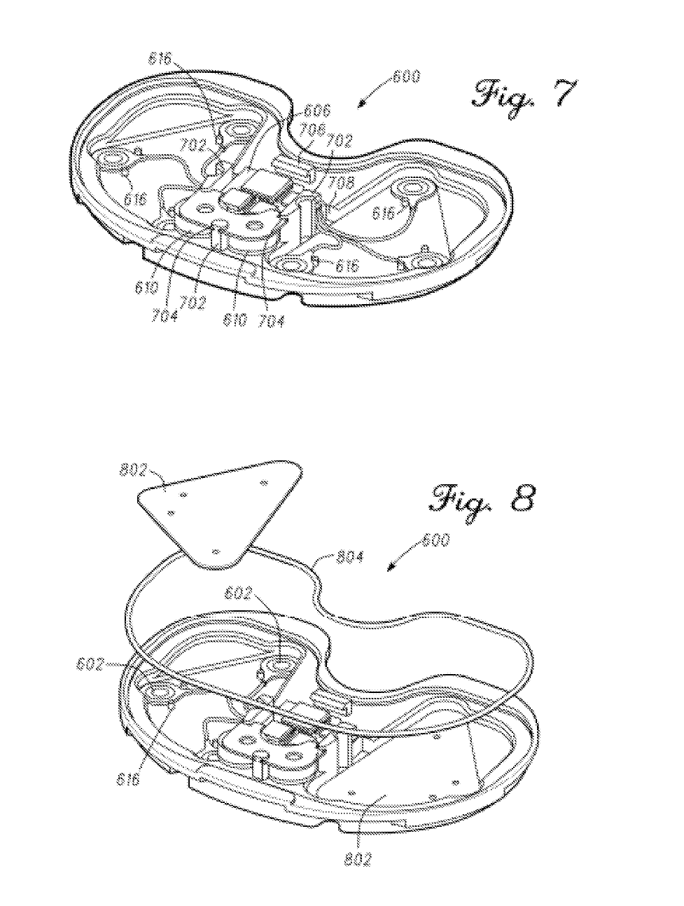

FIG. 7 illustrates the lower support structure 600 with the sensors 602 located in cavities 620 and 622 in accordance with an example embodiment. Electronic circuitry 614 is located centrally between each knee compartment of lower support structure 600. The placement of electronic circuitry 614 is in an un-loaded or lightly loaded region of the insert. The primary joint loading occurs where the condyle surfaces of the femur contact the articular surfaces. The location of electronic circuitry 614 is between the articular surfaces thereby reducing the likelihood of damage to the components for both intra-operative and long-term implant insert use. The location also minimizes the interconnect distance and routing complexity from electronic circuitry 614 to the multiple sensor locations thereby simplifying manufacturing of the system.

In general, retaining structures 702 position and hold electronic circuitry 614 in place. Retaining structures 702 are located in the un-loaded or lightly loaded region of the insert. In one embodiment, a tab 706 for coupling upper support structure to lower support structure 600 also aids in retaining electronic circuitry 614. The components of electronic circuitry 614 are coupled on the printed circuit board 606 to form the circuit for measuring parameters of the muscular-skeletal system. One or more printed circuit boards can be used as well as having multiple layers of interconnects within a printed circuit board. The printed circuit board 606 is positioned on lower support structure 600 such that the batteries 610 can be retained and coupled for powering the system. Batteries 610 are held in place by power source retainer 612. The power source retainer 612 engages with slots 704 in retaining structures 702. The slots 704 can be positioned on retaining structures 702 such that a compressive force is applied to the batteries when retainer 612 is engaged. The power source retainer 612 can further include interconnect for coupling to terminals of the batteries or to couple to electronic circuitry 614.

Sensors 602 are retained by the sidewall of cavities 620 and 622 in conjunction with retaining structures 616. In one embodiment, sensors 602 are circular in shape. The sensors 602 are positioned at each vertex of triangular shaped cavities 620 and 622. The sidewalls of the cavities 620 and 622 accommodate, align, and aid in the retention of the circular shape of each sensor. The sensors 602 contact pad regions 618 that are raised above a bottom surface of cavities 620 and 622. Sensors 602 have flexible interconnect that couple to electronic circuitry 614. The flexible interconnect overlies the bottom surface of cavities 602 and are routed to electronic circuitry 614. A channel 708 can be formed in the periphery of the central region of lower support structure 600 such that the flexible interconnect can be routed from the bottom surface of cavities 620 and 622 to the electronic circuitry 614. The channel 708 provides access to the electronic circuitry 614 without interfering with movement of the load sensors.

FIG. 8 illustrates load plates 802 in accordance with an example embodiment. Load plates 802 distribute loading to sensors 602 in cavities 620 and 622. More specifically, a load applied to an articular surface of the upper support structure is delivered to an underlying load plate. The load plates 802 comprise a rigid material. In one embodiment, load plates 802 are made of metal such as steel. The underlying load plate distributes the applied load to the three sensors of the corresponding cavity. As mentioned previously, the magnitude of the load measured at each sensor location within a cavity is used to determine the magnitude and location of the applied load to the articular surface.

Load plates 802 are shaped to moveably fit within cavities 620 and 622. Movement of load plates 802 is substantially vertical within cavities 620 and 622 wherein the sensors 602 compress under loading. As shown, load plates 802 are triangular in shape. Load plates 802 include openings for receiving retaining structures 616. Retaining structures 616 aid in aligning the load plates 802 to cavities 620 and 622 to simplify assembly. The retaining structures 616 do not bind or inhibit vertical movement of load plates 802. In one embodiment, load plates 802 are planar. Alternatively, load plates 802 can conform to the shape of the overlying articular surface and posts or other structures seen in the various knee implants. Similarly, pad regions 618 can have a non-planar surface to conform to the overlying articular surface. The sensors 602 such as a film sensor can be conformal.

A seal 804 is placed around the interior periphery of lower support structure 600. The electronic circuitry 614 and sensors 602 are within the bounds of seal 804. The seal 804 contacts a perimeter surface of lower support structure 600 and the upper support structure. A lip around the perimeter of the lower support structure 600 and the upper support structure retains seal 804 during assembly. The seal 804 can be an o-ring seal. The peripheral surface of the lower support structure can have a groove in which a portion of seal 804 is seated for positioning and retention. In one embodiment, seal 804 forms a hermetic seal. An enclosure is formed by attaching the upper support structure to lower support structure 600 where seal 804 isolates the sensors 602 and electronic circuitry 614 from an external environment.

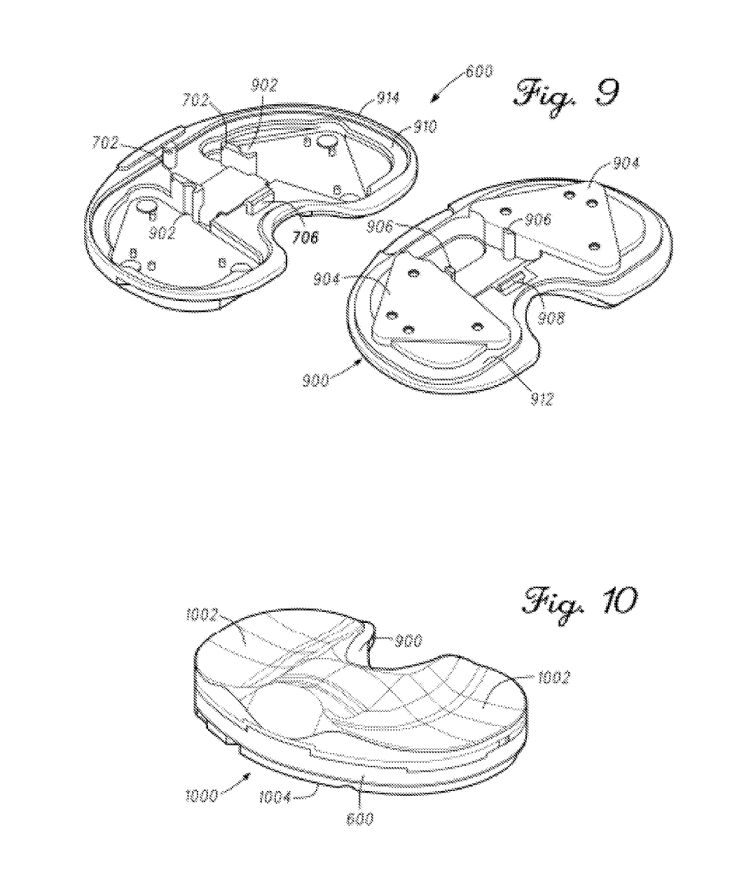

FIG. 9 illustrates lower support structure 600 and upper support structure 900 in accordance with an example embodiment. The lower support structure 600 includes a perimeter surface 910. A lip 914 extends above the perimeter surface 910 at the outer boundary of structure 600. The lip 914 retains a seal that contacts the perimeter surface 910 as disclosed above. The lower support structure 600 includes retaining structures 702 for holding printed circuit board 606 in a fixed position. Retaining structures 702 also aid in the alignment of support structures 600 and 900. A slot 902 is formed in support structures 702 that correspond to guide pins 906 of the upper support structure 900.

Slot 902 of retaining structures 702 has a semi-circular cross-sectional opening. Conversely, guide pins 906 are a column, which for example can have a semi-circular cross-sectional shape. Guide pins 906 align to slots 902 and slideably engage upper support structure 900 to lower support structure 600. An open region or cavity is formed between guide pins 902 in the upper support structure for receiving and housing the electronic circuitry. Upper support structure has surfaces 904 that are shaped similar to load plates 802. Surface 904 underlies and couples to a corresponding articular surface of structure 900. Surfaces 904 contact load plates 802 as upper support structure 900 is mated to lower support. In one embodiment, each surface 904 interfaces to a corresponding load plate 802 when support structures 900 and 600 are attached together.

In one operational example, upper support structure 900 and lower support structure 600 are positioned such that the guide pins 906 are aligned with slots 902. The seal (not shown) is in contact with perimeter surface 910. Structures 600 and 900 are slideably engaged thereby moving the interior surfaces closer together. The attachment mechanism of support structures 900 and 600 comprises a tab 706 and a lock 908. Tab 706 extends from lower support structure 600. Tab 706 is rigid with an extended ledge or lip. Lock 908 aligns with tab 706 and extends from upper support structure 900. In one embodiment, lock 908 is not rigid but can flex or bend. Lock 908 has a canted head with a ledge or lip. The canted head of lock 908 contacts the upper portion of tab 706. The canted head bends lock 908 away from tab 706 as structures 600 and 900 move closer together. A perimeter surface 912 of upper support structure 900 contacts the seal. In one embodiment, the seal is an elastic seal comprising a material such as rubber or a synthetic material such as neoprene. The seal compresses under the pressure applied to couple structures 600 and 900 together. The bending force on lock 908 is released when the ledge surface of lock 908 is co-planar with the ledge surface of tab 706 such that the lock 908 can straighten. An outward elastic force provided by the seal holds the ledge surfaces of lock 908 and 706 together. The upper support structure 900 can be released from the lower support structure 600 by applying a force to bend lock 908 away from tab 706. The structures 600 and 900 are released from one another when the ledge surfaces of tab 706 and lock 908 are no longer in contact with one another.

A method of isolating the electronic circuitry from an external environment is supported by the embodiment disclosed herein. The steps disclosed herein can be performed in any order or combination. In a first step, an enclosure is formed having at least one articular surface and a load bearing surface where a force, pressure, or load is applied by the muscular-skeletal system to the articular and load bearing surfaces. In one embodiment, the enclosure is an insert for allowing articulation of the muscular-skeletal system. In a second step, the electronic circuitry is placed in an un-loaded or lightly loaded region within the enclosure where the insert is substantially equal dimensionally to a final insert. The final insert is a prosthetic component of a joint reconstruction that is implanted into a patient for long-term use. Moreover, natural and artificial joints can sustain high impact force, pressure, or loads under normal use. Placing the electronic circuitry within a region that is un-loaded or lightly loaded region prevents damage and increases reliability for intra-operative or long term applications. In a third step, the enclosure is sealed to isolate the electronic circuitry from the external environment. In one embodiment, the enclosure is hermetically sealed such that the interior and exterior of the insert is sterilized.

In a fourth step, a first support structure is provided. The first support structure has the at least one load bearing surface. The first support structure further includes a surface that is un-loaded or lightly loaded. In the example, the first support structure has two articular or load-bearing surfaces. Between the two articular surfaces is an un-loaded or lightly loaded surface. As disclosed herein the primary loading on the insert occurs between the condyles of the femoral prosthetic component and the articular surfaces of the first support structure. In a fifth step, a second support structure is provided having a load bearing surface. In the example, the load bearing surface of the second support structure interfaces with a tibial prosthetic component. The loading on the insert is compressive such that it occurs across articular and load-bearing surfaces. In the example, the loading is distributed over a much larger surface area between the load bearing surface and tibial prosthetic component than between the combined areas of the condyles to articular surfaces. Thus, the loading on the load-bearing surface is less than the loading on the articular surfaces of the insert. In the example, the loading on the load-bearing surface is substantially less than the loading on the articular surfaces.

In a sixth step, the first and second support structures are coupled together such that the electronic circuitry is located underlying the un-loaded or lightly loaded surface of the first support structure. Coupling the first and second support structures together forms an enclosure for housing the sensors and electronic circuitry for measuring parameters of the muscular-skeletal system. In a seventh step, the electronic circuitry is retained by one or more retaining features within the enclosure. In the example, the electronic circuitry and power source are mounted on a printed circuit board. The second support structure has a surface corresponding to the unloaded or lightly loaded surface of the first support structure. Retaining features extend from the surface of the second support structure to retain and locate the printed circuit board in a position that underlies the un-loaded or lightly loaded surface of the first support structure. In an eighth step, a plurality of sensors are coupled between the articular surface and the load-bearing surface of the enclosure. In one embodiment, the sensors measure a force, pressure, or load applied across the articular and load-bearing surfaces. The sensors are located at predetermined locations in relation to the articular surface to identify a position where the force, pressure, or load is applied. In a ninth step, the insert is disposed of after using the insert intra-operatively.

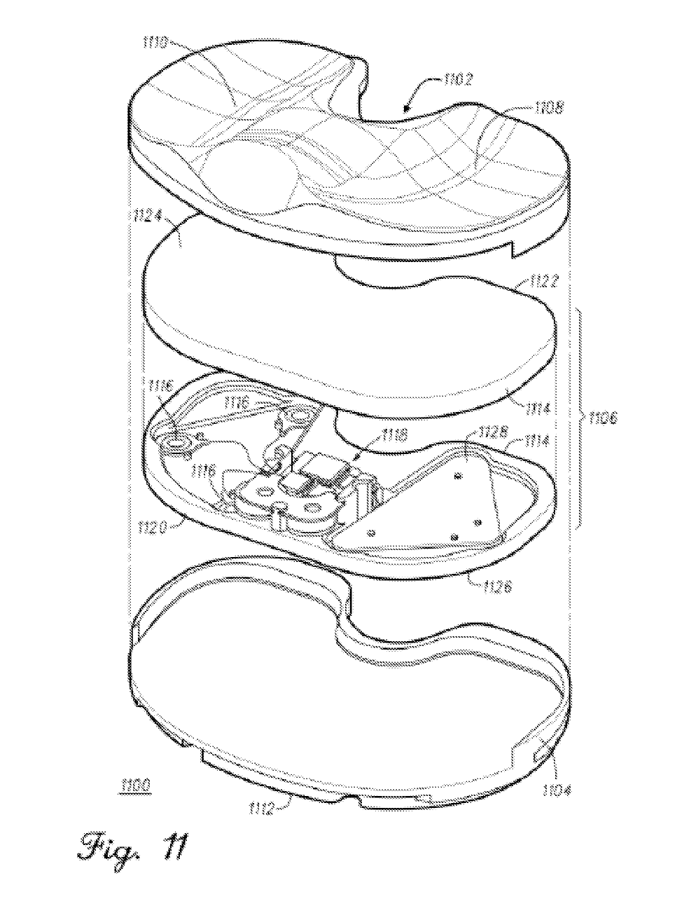

FIG. 10 illustrates attached components for an insert 1000 in accordance with an example embodiment. Insert 1000 comprises lower support structure 600, upper support structure 900, and shim 1004. The insert system includes removable shims of different heights for aiding in the selection of an appropriate final insert. Shim 1004 attaches to lower support structure 600. Insert 1000 is an active device having electronic circuitry, a power source, communication circuitry, and sensors within the enclosure formed by support structures 600 and 900. Upper support structure 900 has articular surfaces 1002 for allowing articulation of the muscular-skeletal system. The sensors underlie the articular surfaces 1002 as disclosed hereinabove. Measurements are taken and sent via wireless communication to an external receiver. As shown, insert 1000 is dimensionally substantially equal to a final insert when used intra-operatively. In at least one embodiment, insert 1000 is a final insert for use in taking parameter measurements on the joint status. Thus, insert 1000 can be used similarly to passive inserts while providing quantitative data for assessing aspects of the muscular-skeletal system or prosthetic components used therein.

FIG. 11 illustrates components of insert sensing device 1100 in accordance with an example embodiment. Insert sensing device 1100 (or insert 1100) comprises an upper support structure 1102, a lower support structure 1104, and a sensing module 1106. For illustration purposes, insert sensing device 1100 is shown as a knee insert for a total knee reconstruction. Insert sensing device 1100 can be used in other joint inserts such as spine, hip, shoulder, ankle, and others for parameter measurement device of muscular-skeletal system. Upper support structure 1102 has articular surfaces 1108 and 1110. Articular surfaces 1108 and 1110 interface with the condylar surfaces of a femur to allow leg motion. Lower support structure 1104 has a load bearing surface 1112. The load-bearing surface 1112 interfaces with the tibia or a prosthetic tibial component. Although not shown, the insert sensing device 1100 can further include shims for height adjustment as disclosed herein. The shims attach to the load bearing surface 1112 and are removable.

The support structures 1102 and 1104 include alignment structures to aid in positioning the structures to one another during an attachment process. The support structures 1102 and 1104 can have corresponding tabs and slots for attachment as disclosed herein. The support structures 1102 and 1104 can be temporarily or permanently coupled together. In the example, support structures 1102 and 1104 form an enclosure. The enclosure includes a slot or opening to receiving a sensing module 1106. In the example, the slot is in a sidewall of the insert sensing device 1100. The slot opens into a cavity within the support structures 1102 and 1104. In particular, the cavity underlies the articular surfaces 1108 and 1110.

The measurement module 1106 is a self-contained sensing unit for measurement of the muscular-skeletal system. In the example, the parameter being measured is a force, pressure, or load applied to the articular surfaces 1108 and 1110. Measurement module 1106 includes a housing 1114, sensors 1116, pad regions, load plates, a power source, an antenna, and electronic circuitry 1118. The measurement module 1106 as shown includes a power source such as a battery to power electronic circuitry 1118. The measurement module 1106 further includes a housing 1114 for isolating electronic circuitry 1118 and sensors 1116 from an external environment. The housing 1114 comprises a lower support structure 1120, and an upper support structure 1122. The lower support structure 1120 has a major surface 1126 that interfaces with an interior major surface of support structure 1104. Similarly, the upper support structure 1122 has a major surface 1124 that interfaces with an interior major surface of support structure 1102. The electronic circuitry 1118 and sensors 1116 have a layout architecture similar to that shown in FIG. 7. A load plate is removed to show sensors 1116. A load plate 1128 within measurement module 1106 couples to upper support structure 1122. In one embodiment, a load applied to the articular surface 1108 is transferred through support structures 1102 and 1122 to the load plate 1128 corresponding to a knee compartment of the knee joint. The interior surface of support structure 1122 interfaces to the load plate 1128 to transfer a force, pressure, or load to the underlying sensors (not shown). Sensors underlying load plate 1128 measure the applied force at different predetermined positions. In one embodiment, three sensors 1106 underlie each load plate of each compartment to facilitate identifying a location of where the load is applied to an articular surface. The electronic circuitry 1118 is operatively coupled to the sensors, which produces data corresponding to the force, pressure, or load magnitude as well as the position where the load is applied to the articular surface.

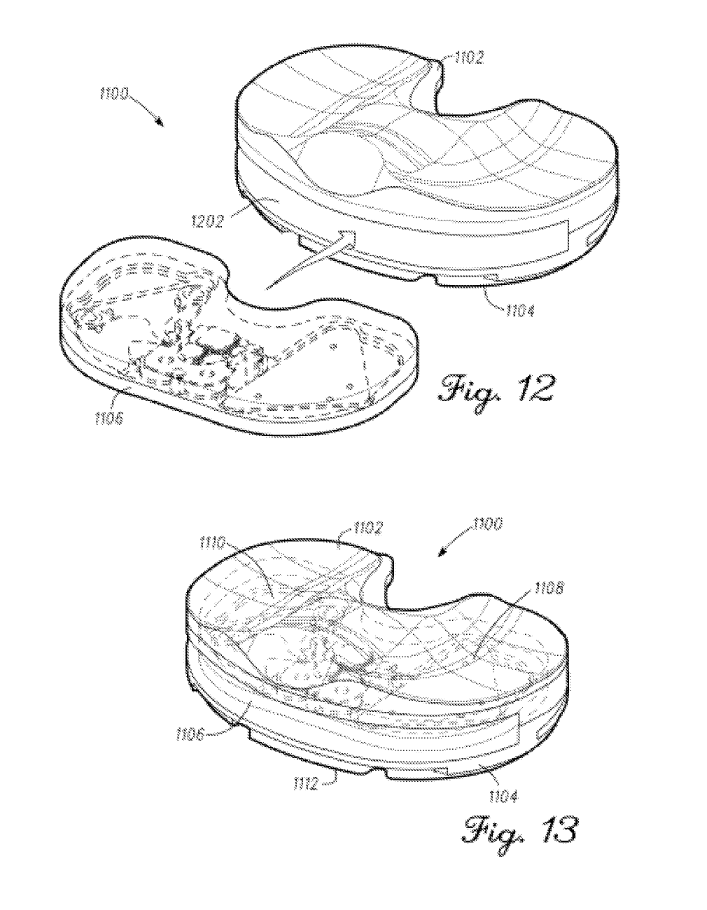

FIG. 12 illustrates a slot 1202 in the insert sensing device 1100 in accordance with an example embodiment. Support structures 1102 and 1104 are coupled together permanently or temporarily. As shown, slot 1202 is an opening in the sidewall of insert sensing device 1100. Slot 1202 provides access to a cavity within support structures 1102 and 1104. The measurement module 1106 is inserted into the slot 1202 to perform measurements on the muscular-skeletal system. In one embodiment, the slot 1202 is approximately parallel with the load bearing surface 1112. The measurement module 1106 slideably engages through the slot of insert sensing device 1100 into the cavity. The sensors underlie the articular surfaces 1108 and 1110 when sensing module 1106 is fully inserted into the cavity. The electronic circuitry within measurement module 1106 is located in a region of insert sensing device 1100 that is unloaded or lightly loaded. In particular, the electronic circuitry is located between the articular surfaces 1108 and 1110 when placed in the cavity.

The lower surface of measurement module 1106 interfaces with the interior major surface of lower support structure 1104. The upper surface of measurement module 1106 has two major surfaces corresponding to articular surfaces 1108 and 1110 of the upper support structure 1104. Each upper surface of measurement module 1106 interfaces with a corresponding interior surface of upper support structure 1102. An applied load to each articular surface results in the transfer of the loading to sensors 1116 in the module 1106. The measurement module 1106 can measure the magnitude of the loading and the position of the applied load on the corresponding articular surface. The measurements are transmitted via wireless communication to an external receiver.

The use of measurement module 1106 allows a common module to be used with different size inserts. The measurement module 1106 can be activated or enabled prior to insertion into device 1100. The module 1106 can be tested and communicate with a remote receiver while in the sterilized package, removed from packaging, and inserted in the insert sensing device 1100. The module 1106 can be removed from the device 1100 and disposed of after being used intra-operatively to aid in the installation of prosthetic components.

FIG. 13 illustrates the measurement module 1106 inserted in the slot of the insert sensing device 1100 in accordance with an example embodiment. In one embodiment, the measurement module 1106 fits within the bounds of upper and lower support structures 1102 and 1104. In the example, the major surfaces 1124 and 1126 of measurement module 1106 are in intimate contact with the interior surfaces of support structures 1102 and 1104. The measurement module 1106 slideably engages until it is positioned in a predetermined location. Physical, auditory, visual, or other feedback can be provided to the user to indicate the module 1106 is positioned correctly. The major surfaces 1124 and 1126 of measurement module 1106 respectively interface with the interior surface of support structure 1102 and the interior surface of support structure 1104 in the cavity coupled to slot 1202. In particular, the sensors of each compartment of measurement module 1106 couple to and underlie a corresponding articular surface to which a force, pressure, or load is applied. The force, pressure, or load couples through the support structure 1102, the support structure 1122, and a load plate that is coupled to at least one sensor. In one embodiment, the electronic circuitry in measurement module 1106 is located centrally to the major exposed surface of upper support structure 1102 in a region that is un-loaded or lightly loaded by the muscular-skeletal system. The insert sensing device 1100 is dimensionally substantially equal to a final insert. The insert sensing device 1100 can be used intra-operatively to aid in the fitting of prosthetic components or as a final insert.

A method of measuring a parameter of the muscular skeletal system is supported by the embodiment disclosed herein. The steps disclosed can be performed in any order or combination. The method can take more or less steps than that disclosed. In a first step, an insert is provided. The insert has at least one articulating surface and a load-bearing surface. The insert allows articulation of the muscular-skeletal system when inserted therein. In a second step, a measurement module is inserted through a slot in the insert. The measurement module includes electronic circuitry, sensors, and a power source to measure the parameter of interest. In one embodiment, the slot is in a sidewall of the insert.

In a third step, major surfaces of the measurement module slideably interface with interior surfaces of the insert. The slot in the insert opens into a cavity within the interior of the insert. The interior surfaces of the insert correspond to major surfaces of the cavity. In a fourth step, the measurement module is positioned to a predetermined location within the cavity. In the example, the parameter being measured is a force, pressure, or load applied by the muscular-skeletal system. A compressive force is applied across the articular surface and the load-bearing surface of the insert. In the knee example, the load is applied to the articular surface of each knee compartment and supported by the entire load-bearing surface. The measurement module is positioned such that a first major surface of the measurement module couples to the articular surfaces of the insert. More specifically, dedicated sensors within the measurement module underlie and are coupled to a corresponding articular surface to measure the force, pressure, or load applied thereto. Similarly, a second major surface of the measurement module couples to the load-bearing surface of the insert.

In a fifth step, a location where the parameter is applied to the articular surface is determined. As mentioned hereinabove, three sensors in predetermined locations couple to a corresponding articular surface. The predetermined locations correspond to areas, regions, or locations of the articular surface. The magnitude and differentials of the measured force, pressure, or load in conjunction with the predetermined locations of the sensors are used to identify where the parameter is applied and the magnitude of the force, pressure, or load. In the example, the parameter measurements are used to optimally fit prosthetic components including an insert in a joint of the muscular-skeletal system. In the example, the measurements determine if the loading, load position, and the balance between knee compartments corresponds to best-known practices for knee joint reconstruction. The measurements can indicate that the height or thickness is insufficient for the reconstructed joint. Alternatively, the measurements can indicate that the bone preparation for receiving prosthetic components is not aligned appropriately to a mechanical axis of the muscular-skeletal system. For example, the measurements indicate that the pressure applied to the articular surface is lower than optimal. The insert is then removed from the joint and adjusted in height for a subsequent fitting. The measuring module does not have to be removed from the insert.

In a sixth step, a shim is added to the insert to modify the height or thickness of the shim. In the example, the added height increases the force, pressure, or load applied by the muscular-skeletal system when the insert is reinserted. The shim and insert comprise a predetermined height that corresponds to an available final insert. In a seventh step the insert with the shim is inserted in the joint. The parameters as disclosed above are then measured with the insert having the new height or thickness. The process of replacing shims can be repeated until an optimal fit is achieved. It should be noted that the surgeon could perform adjustments to the muscular-skeletal system that change the measured parameters. The measurement module measures the changes allowing the surgeon to see the results of the modifications in real-time. For example, the surgeon can adjust the balance between compartments or the magnitude of the applied load using a technique such as soft tissue tensioning. The insert which is substantially dimensionally equal to a final insert allows access to regions for the tensioning procedure.

The insert is then removed from the reconstructed joint. A final insert that is substantially dimensionally equal to the intra-operative insert is placed in the joint. The final insert can have parameter measurement circuitry as disclosed herein. The loading and balance on the articular surfaces of the final insert is substantially equal to that measured by the intra-operative insert. In a seventh step, the measurement module is removed through the slot of the insert. In one embodiment, the measurement module has a power source that is sufficient for only a one surgical procedure. Moreover, the measurement module cannot be opened to replace the power source. The measurement module is low-cost where it can be a disposable item that is used only for a single operation. This eliminates problems associated with re-sterilization processes and patient infection. In an eighth step, the measurement module is disposed of after the surgical procedure is completed or when the parameters have been measured and the final insert selected. Alternatively, the entire insert can be disposed of with the measurement module such that the measurement module is not removed from the insert.

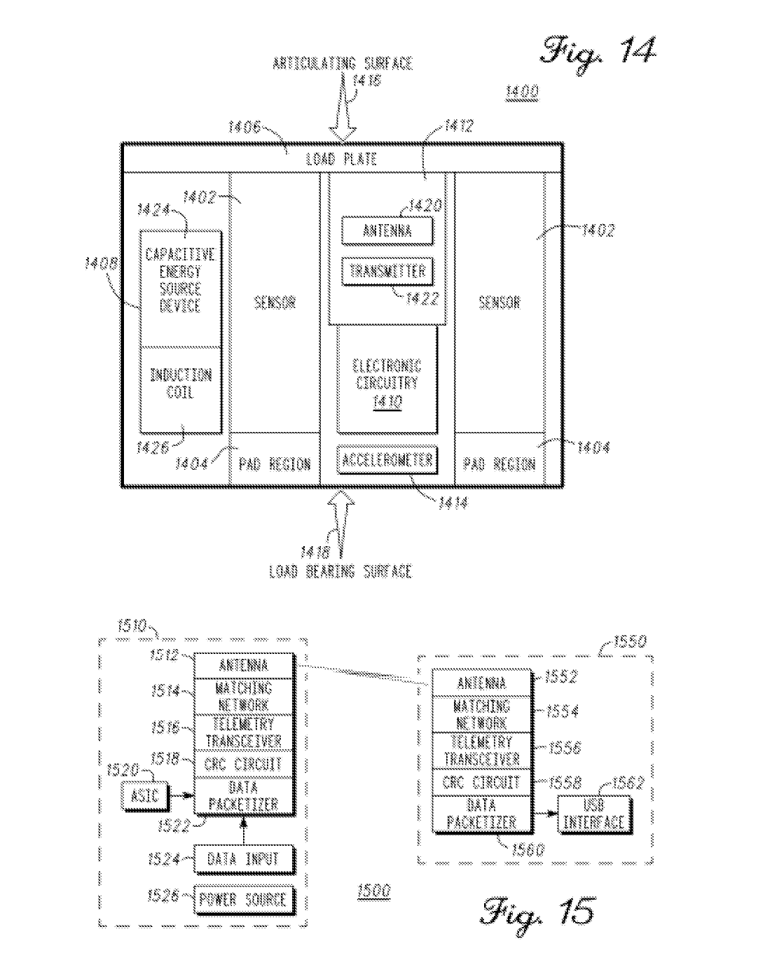

FIG. 14 illustrates components of an insert sensing device 1400 in accordance with an example embodiment. It should be noted that insert sensing device 1400 could comprise more or less than the number of components shown. Insert sensing device 1400 is a prosthetic component allowing parameter measurement and articulation of the muscular-skeletal system. As illustrated, the insert sensing device 1400 includes one or more sensors 1402, a pad region 1404, a load plate 1406, a power source 1408, electronic circuitry 1410, a transceiver 1412, and an accelerometer 1414. In a non-limiting example, the insert sensing device 1400 can measure an applied compressive force.

The sensors 1402 can be positioned, engaged, attached, or affixed to the contact surfaces 1416 and 1418. In at least one example embodiment, contact surfaces 1416 and 1418 are load-bearing surfaces. In the example of a knee insert, surface 1416 is a load bearing articular surface that contacts a femoral condyle that together allows movement of the muscular-skeletal system. Contact surface 1418 is a load bearing surface. In the example, contact surface 1418 contacts a tibial surface in a fixed position. Surfaces 1416 and 1418 can move and tilt with changes in applied load actions, which can be transferred to the sensors 1402 and measured by the electronic circuitry 1410. The electronic circuitry 1410 measures physical changes in the sensors 1401 to determine parameters of interest, for example a magnitude, distribution and direction of forces acting on the contact surfaces 1416 and 1418. The insert sensing device 1400 is powered by an internal power source 1408.

As one example, sensors 1402 can comprise an elastic or compressible propagation structure between a first transducer and a second transducer. The transducers can be an ultrasound (or ultrasonic) resonator, and the elastic or compressible propagation structure can be an ultrasound waveguide. The electronic circuitry 1410 is electrically coupled to the transducers to translate changes in the length (or compression or extension) of the compressible propagation structure to parameters of interest, such as force. The system measures a change in the length of the compressible propagation structure (e.g., waveguide) responsive to an applied force and converts this change into electrical signals, which can be transmitted via the transceiver 1412 to convey a level and a direction of the applied force. For example, the compressible propagation structure has known and repeatable characteristics of the applied force versus the length of the waveguide. Precise measurement of the length of the waveguide using ultrasonic signals can be converted to a force using the known characteristics.

Sensors 1402 are not limited to waveguide measurements of force, pressure, or load sensing. In yet other arrangements, the sensors can include piezoelectric, capacitive, optical or temperature sensors to provide other parameter measurements. Moreover, for force, pressure, or load sensing, other sensor types such as piezo-resistive sensors, mems devices, strain gauges, and mechanical sensors can be used in conjunction with the electronic circuitry 1410. In one embodiment, much of the electronic circuitry 1410 is integrated onto an application specific integrated circuit (ASIC). The ASIC reduces power consumption and form factor while increasing the sensing capabilities of device 1400. In particular, electronic circuitry 1410 includes multiple inputs, outputs, and input/outputs thereby allowing both serial and parallel data transfer. The ASIC also incorporates digital control logic to manage control functions of device 1400. The electronic circuitry 1410 or ASIC incorporates A/D and D/A circuitry (not shown) to digitize current and voltage output from these types of sensing components.

The accelerometer 1414 can measure acceleration and static gravitational pull. Accelerometer 1414 can be single-axis and multi-axis accelerometer structures that detect magnitude and direction of the acceleration as a vector quantity. Accelerometer 1414 can also be used to sense orientation, vibration, impact and shock. The electronic circuitry 1410 in conjunction with the accelerometer 1414 and sensors 1402 can measure parameters of interest (e.g., distributions of load, force, pressure, displacement, movement, rotation, torque and acceleration) relative to orientations of insert sensing device 1400 with respect to a reference point. In such an arrangement, spatial distributions of the measured parameters relative to a chosen frame of reference can be computed and presented for real-time display.

The transceiver 1412 comprises a transmitter 1422 and an antenna 1420 to permit wireless operation and telemetry functions. In various embodiments, the antenna 1420 can be configured by design as an integrated loop antenna. The integrated loop antenna is configured at various layers and locations on a printed circuit board having other electrical components mounted thereto. Once initiated the transceiver 1412 can broadcast the parameters of interest in real-time. The telemetry data can be received and decoded with various receivers, or with a custom receiver. The wireless operation can eliminate distortion of, or limitations on, measurements caused by the potential for physical interference by, or limitations imposed by, wiring and cables coupling the sensing module with a power source or with associated data collection, storage, display equipment, and data processing equipment.

The transceiver 1412 receives power from the power source 1408 and can operate at low power over various radio frequencies by way of efficient power management schemes, for example, incorporated within the electronic circuitry 1410. As one example, the transceiver 1412 can transmit data at selected frequencies in a chosen mode of emission by way of the antenna 1420. The selected frequencies can include, but are not limited to, ISM bands recognized in International Telecommunication Union regions 1, 2 and 3. A chosen mode of emission can be, but is not limited to, Gaussian Frequency Shift Keying, (GFSK), Amplitude Shift Keying (ASK), Phase Shift Keying (PSK), Minimum Shift Keying (MSK), Frequency Modulation (FM), Amplitude Modulation (AM), or other versions of frequency or amplitude modulation (e.g., binary, coherent, quadrature, etc.).

The antenna 1420 can be integrated with components of the sensing module to provide the radio frequency transmission. The antenna 1420 and electronic circuitry 1410 are mounted and coupled to form a circuit using wire traces on a printed circuit board. The antenna 1420 can further include a matching network for efficient transfer of the signal. This level of integration of the antenna and electronics enables reductions in the size and cost of wireless equipment. Potential applications may include, but are not limited to any type of short-range handheld, wearable, or other portable communication equipment where compact antennas are commonly used. This includes disposable modules or devices as well as reusable modules or devices and modules or devices for long-term use.

The power source 1408 provides power to electronic components of the insert sensing device 1400. In one embodiment, the power source 1408 can be charged by wired energy transfer, short-distance wireless energy transfer or a combination thereof. External power sources for providing wireless energy to power source 1408 can include, but are not limited to, a battery or batteries, an alternating current power supply, a radio frequency receiver, an electromagnetic induction coil, energy harvesting, magnetic resonance charging, a photoelectric cell or cells, a thermocouple or thermocouples, or an ultrasound transducer or transducers. By way of power source 1408, insert sensing device 1400 can be operated with a single charge until the internal energy is drained. It can be recharged periodically to enable continuous operation. The power source 1408 can further utilize power management techniques for efficiently supplying and providing energy to the components of device 1400 to facilitate measurement and wireless operation. Power management circuitry can be incorporated on the ASIC to manage both the ASIC power consumption as well as other components of the system.

The power source 1408 minimizes additional sources of energy radiation required to power the sensing module during measurement operations. In one embodiment, as illustrated, the energy storage 1408 can include a capacitive energy storage device 1424 and an induction coil 1426. The external source of charging power can be coupled wirelessly to the capacitive energy storage device 1424 through the electromagnetic induction coil or coils 1426 by way of inductive charging. The charging operation can be controlled by power management systems designed into, or with, the electronic circuitry 1410. For example, during operation of electronic circuitry 1410, power can be transferred from capacitive energy storage device 1410 by way of efficient step-up and step-down voltage conversion circuitry. This conserves operating power of circuit blocks at a minimum voltage level to support the required level of performance. An alternative to the capacitive energy storage device 1424 is a rechargeable battery disclosed hereinabove that could be recharged wirelessly as described herein.

In one configuration, the external power source can further serve to communicate downlink data to the transceiver 1412 during a recharging operation. For instance, downlink control data can be modulated onto the wireless energy source signal and thereafter demodulated from the induction coil 1426 by way of electronic circuitry 1410. This can serve as a more efficient way for receiving downlink data instead of configuring the transceiver 1412 for both uplink and downlink operation. As one example, downlink data can include updated control parameters that the device 1400 uses when making a measurement, such as external positional information, or for recalibration purposes. It can also be used to download a serial number or other identification data.

The electronic circuitry 1410 manages and controls various operations of the components of the sensing module, such as sensing, power management, telemetry, and acceleration sensing. It can include analog circuits, digital circuits, integrated circuits, discrete components, or any combination thereof. In one arrangement, it can be partitioned among integrated circuits and discrete components to minimize power consumption without compromising performance. Partitioning functions between digital and analog circuit enhances design flexibility and facilitates minimizing power consumption without sacrificing functionality or performance. Accordingly, the electronic circuitry 1410 can comprise one or more integrated circuits or ASICs, for example, specific to a core signal processing algorithm.

In another arrangement, the electronic circuitry 1410 can comprise a controller such as a programmable processor, a Digital Signal Processor (DSP), a microcontroller, or a microprocessor, with associated storage memory and logic. The controller can utilize computing technologies with associated storage memory such a Flash, ROM, RAM, SRAM, DRAM or other like technologies for controlling operations of the aforementioned components of the sensing module. In one arrangement, the storage memory may store one or more sets of instructions (e.g., software) embodying any one or more of the methodologies or functions described herein. The instructions may also reside, completely or at least partially, within other memory, and/or a processor during execution thereof by another processor or computer system.

The electronics assemblage also supports testability and calibration features that assure the quality, accuracy, and reliability of the completed wireless sensing module or device. A temporary bi-directional coupling assures a high level of electrical observability and controllability of the electronics. The test interconnect also provides a high level of electrical observability of the sensing subsystem, including the transducers, waveguides, and mechanical spring or elastic assembly. Carriers or fixtures emulate the final enclosure of the completed wireless sensing module or device during manufacturing processing thus enabling capture of accurate calibration data for the calibrated parameters of the finished wireless sensing module or device. These calibration parameters are stored within the on-board memory integrated into the electronics assemblage.

Applications for the electronic assembly comprising the sensors 1402 and electronic circuitry 1410 may include, but are not limited to, disposable modules or devices as well as reusable modules or devices and modules or devices for long-term use. In addition to non-medical applications, examples of a wide range of potential medical applications may include, but are not limited to, implantable devices, modules within implantable devices, intra-operative implants or modules within intra-operative implants or trial inserts, modules within inserted or ingested devices, modules within wearable devices, modules within handheld devices, modules within instruments, appliances, equipment, or accessories of all of these, or disposables within implants, trial inserts, inserted or ingested devices, wearable devices, handheld devices, instruments, appliances, equipment, or accessories to these devices, instruments, appliances, or equipment.

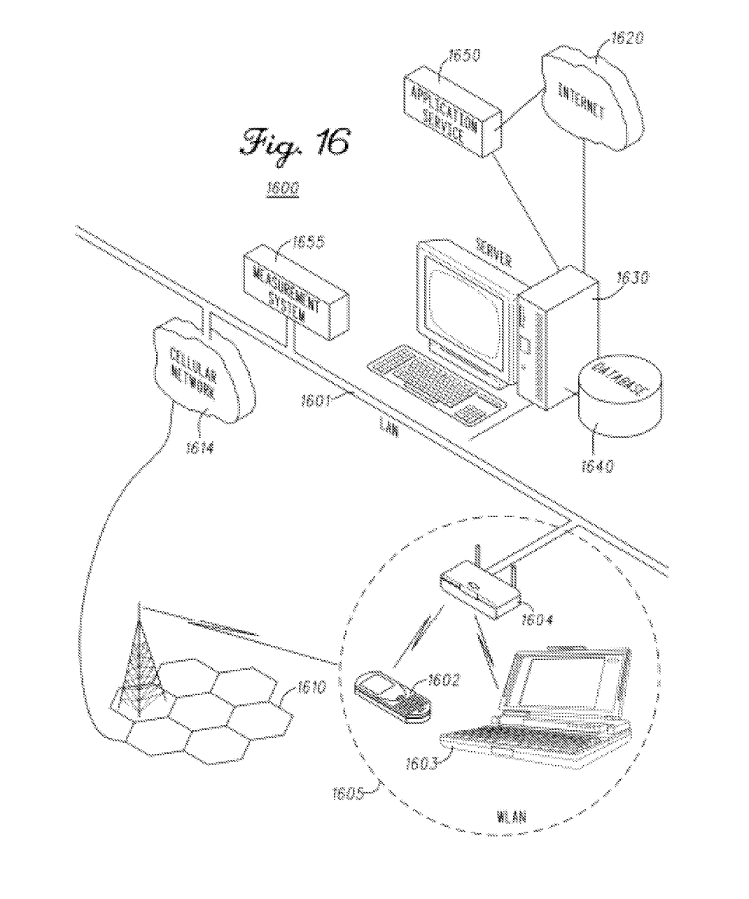

FIG. 15 illustrates a communications system 1500 for short-range telemetry in accordance with an example embodiment. As illustrated, the communications system 1500 comprises medical device communications components 1510 in a prosthetic component and receiving system communications in a processor based system. In one embodiment, the receiving system communications are in or coupled to a computer or laptop computer that is external to the sterile field of the operating room. The surgeon can view the laptop screen or a display coupled to the computer while performing surgery. The medical device communications components 1510 are operatively coupled to include, but not limited to, the antenna 1512, a matching network 1514, the telemetry transceiver 1516, a CRC circuit 1518, a data packetizer 1522, a data input 1524, a power source 1526, and an application specific integrated circuit (ASIC) 1520. The medical device communications components 1510 may include more or less than the number of components shown and are not limited to those shown or the order of the components.

The receiving station communications components comprise an antenna 1542, a matching network 1554, the telemetry transceiver 1556, the CRC circuit 1558, the data packetizer 1560, and optionally a USB interface 1562. Notably, other interface systems can be directly coupled to the data packetizer 1560 for processing and rendering sensor data.

In general, the electronic circuitry is operatively coupled to one or more sensors of the prosthetic component. In one embodiment, the data generated by the one or more sensors can comprise a voltage or current value from a mems structure, piezo-resistive sensor, strain gauge, mechanical sensor or other sensor type that is used to measure a parameter of the muscular-skeletal system. The data packetizer 1522 assembles the sensor data into packets; this includes sensor information received or processed by ASIC 1520. The ASIC 1520 can comprise specific modules for efficiently performing core signal processing functions of the medical device communications components 1510. The ASIC 1520 provides the further benefit of reducing the form factor of insert sensing device to meet dimensional requirements for integration into temporary or permanent prosthetic components.

The CRC circuit 1518 applies error code detection on the packet data. The cyclic redundancy check is based on an algorithm that computes a checksum for a data stream or packet of any length. These checksums can be used to detect interference or accidental alteration of data during transmission. Cyclic redundancy checks are especially good at detecting errors caused by electrical noise and therefore enable robust protection against improper processing of corrupted data in environments having high levels of electromagnetic activity. The telemetry transceiver 1516 then transmits the CRC encoded data packet through the matching network 1514 by way of the antenna 1512. The matching networks 1514 and 1554 provide an impedance match for achieving optimal communication power efficiency.

The receiving system communications components 1550 receive transmission sent by medical device communications components 1510. In one embodiment, telemetry transceiver 1516 is operated in conjunction with a dedicated telemetry transceiver 1556 that is constrained to receive a data stream broadcast on the specified frequencies in the specified mode of emission. The telemetry transceiver 1556 by way of the receiving station antenna 1552 detects incoming transmissions at the specified frequencies. The antenna 1552 can be a directional antenna that is directed to a directional antenna of components 1510. Using at least one directional antenna can reduce data corruption while increasing data security by further limiting where the data is radiated. A matching network 1554 couples to antenna 1552 to provide an impedance match that efficiently transfers the signal from antenna 1552 to telemetry transceiver 1556. Telemetry transceiver 1556 can reduce a carrier frequency in one or more steps and strip off the information or data sent by components 1510. Telemetry transceiver 1556 couples to CRC circuit 1558. CRC circuit 1558 verifies the cyclic redundancy checksum for individual packets of data. CRC circuit 1558 is coupled to data packetizer 1560. Data packetizer 1560 processes the individual packets of data. In general, the data that is verified by the CRC circuit 1558 is decoded (e.g., unpacked) and forwarded to an external data processing device, such as an external computer, for subsequent processing, display, or storage or some combination of these.

The telemetry transceiver 1556 is designed and constructed to operate on very low power such as, but not limited to, the power available from the powered USB port 1562, or a battery. In another embodiment, the telemetry transceiver 1556 is designed for use with a minimum of controllable functions to limit opportunities for inadvertent corruption or malicious tampering with received data. The telemetry transceiver 1556 can be designed and constructed to be compact, inexpensive, and easily manufactured with standard manufacturing processes while assuring consistently high levels of quality and reliability.

In one configuration, the communication system 1500 operates in a transmit-only operation with a broadcasting range on the order of a few meters to provide high security and protection against any form of unauthorized or accidental query. The transmission range can be controlled by the transmitted signal strength, antenna selection, or a combination of both. A high repetition rate of transmission can be used in conjunction with the Cyclic Redundancy Check (CRC) bits embedded in the transmitted packets of data during data capture operations thereby enabling the receiving system to discard corrupted data without materially affecting display of data or integrity of visual representation of data, including but not limited to measurements of load, force, pressure, displacement, flexion, attitude, and position within operating or static physical systems.

By limiting the operating range to distances on the order of a few meters the telemetry transceiver 1516 can be operated at very low power in the appropriate emission mode or modes for the chosen operating frequencies without compromising the repetition rate of the transmission of data. This mode of operation also supports operation with compact antennas, such as an integrated loop antenna. The combination of low power and compact antennas enables the construction of, but is not limited to, highly compact telemetry transmitters that can be used for a wide range of non-medical and medical applications.

The transmitter security as well as integrity of the transmitted data is assured by operating the telemetry system within predetermined conditions. The security of the transmitter cannot be compromised because it is operated in a transmit-only mode and there is no pathway to hack into medical device communications components. The integrity of the data is assured with the use of the CRC algorithm and the repetition rate of the measurements. The risk of unauthorized reception of the data is minimized by the limited broadcast range of the device. Even if unauthorized reception of the data packets should occur there are counter measures in place that further mitigate data access. A first measure is that the transmitted data packets contain only binary bits from a counter along with the CRC bits. A second measure is that no data is available or required to interpret the significance of the binary value broadcast at any time. A third measure that can be implemented is that no patient or device identification data is broadcast at any time.

The telemetry transceiver 1516 can also operate in accordance with some FCC regulations. According to section 18.301 of the FCC regulations the ISM bands within the USA include 6.78, 13.56, 27.12, 30.68, 915, 2450, and 5800 MHz as well as 24.125, 61.25, 122.50, and 245 GHz. Globally other ISM bands, including 433 MHz, are defined by the International Telecommunications Union in some geographic locations. The list of prohibited frequency bands defined in 18.303 are “the following safety, search and rescue frequency bands is prohibited: 490-510 kHz, 2170-2194 kHz, 8354-8374 kHz, 121.4-121.6 MHz, 156.7-156.9 MHz, and 242.8-243.2 MHz.” Section 18.305 stipulates the field strength and emission levels ISM equipment must not exceed when operated outside defined ISM bands. In summary, it may be concluded that ISM equipment may be operated worldwide within ISM bands as well as within most other frequency bands above 9 KHz given that the limits on field strengths and emission levels specified in section 18.305 are maintained by design or by active control. As an alternative, commercially available ISM transceivers, including commercially available integrated circuit ISM transceivers, may be designed to fulfill these field strengths and emission level requirements when used properly.

In one configuration, the telemetry transceiver 1516 can also operate in unlicensed ISM bands or in unlicensed operation of low power equipment, wherein the ISM equipment (e.g., telemetry transceiver 1516) may be operated on ANY frequency above 9 kHz except as indicated in Section 18.303 of the FCC code.

Wireless operation eliminates distortion of, or limitations on, measurements caused by the potential for physical interference by, or limitations imposed by, wiring and cables coupling the wireless sensing module or device with a power source or with data collection, storage, or display equipment. Power for the sensing components and electronic circuits is maintained within the wireless sensing module or device on an internal energy storage device. This energy storage device is charged with external power sources including, but not limited to, a battery or batteries, super capacitors, capacitors, an alternating current power supply, a radio frequency receiver, an electromagnetic induction coil, a photoelectric cell or cells, a thermocouple or thermocouples, or an ultrasound transducer or transducers. The wireless sensing module may be operated with a single charge until the internal energy source is drained or the energy source may be recharged periodically to enable continuous operation. The embedded power supply minimizes additional sources of energy radiation required to power the wireless sensing module or device during measurement operations. Telemetry functions are also integrated within the wireless sensing module or device. Once initiated the telemetry transmitter continuously broadcasts measurement data in real time. Telemetry data may be received and decoded with commercial receivers or with a simple, low cost custom receiver.

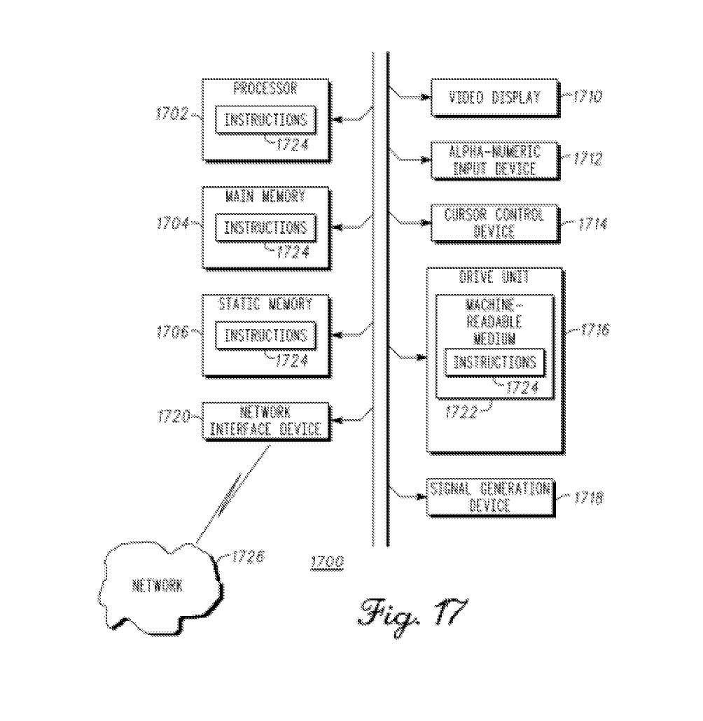

FIG. 16 illustrates a communication network 1600 for measurement and reporting in accordance with an example embodiment. Briefly, the communication network 1600 expands broad data connectivity to other devices or services. As illustrated, the measurement and reporting system 1655 can be communicatively coupled to the communications network 1600 and any associated systems or services.

As one example, the measurement system 1655 can share its parameters of interest (e.g., angles, load, balance, distance, alignment, displacement, movement, rotation, and acceleration) with remote services or providers, for instance, to analyze or report on surgical status or outcome. This data can be shared for example with a service provider to monitor progress or with plan administrators for surgical monitoring purposes or efficacy studies. The communication network 1600 can further be tied to an Electronic Medical Records (EMR) system to implement health information technology practices. In other embodiments, the communication network 1600 can be communicatively coupled to HIS Hospital Information System, HIT Hospital Information Technology and HIM Hospital Information Management, EHR Electronic Health Record, CPOE Computerized Physician Order Entry, and CDSS Computerized Decision Support Systems. This provides the ability of different information technology systems and software applications to communicate, to exchange data accurately, effectively, and consistently, and to use the exchanged data.

The communications network 1600 can provide wired or wireless connectivity over a Local Area Network (LAN) 1601, a Wireless Local Area Network (WLAN) 1605, a Cellular Network 1614, and/or other radio frequency (RF) system. The LAN 1601 and WLAN 1605 can be communicatively coupled to the Internet 1620, for example, through a central office. The central office can house common network switching equipment for distributing telecommunication services. Telecommunication services can include traditional POTS (Plain Old Telephone Service) and broadband services such as cable, HDTV, DSL, VoIP (Voice over Internet Protocol), IPTV (Internet Protocol Television), Internet services, and so on.

The communication network 1600 can utilize common computing and communications technologies to support circuit-switched and/or packet-switched communications. Each of the standards for Internet 1620 and other packet switched network transmission (e.g., TCP/IP, UDP/IP, HTML, HTTP, RTP, MMS, SMS) represent examples of the state of the art. Such standards are periodically superseded by faster or more efficient equivalents having essentially the same functions. Accordingly, replacement standards and protocols having the same functions are considered equivalent.

The cellular network 1614 can support voice and data services over a number of access technologies such as GSM-GPRS, EDGE, CDMA, UMTS, WiMAX, 2G, 3G, 4G, WAP, software defined radio (SDR), and other known technologies. The cellular network 1614 can be coupled to base receiver 1610 under a frequency-reuse plan for communicating with mobile devices 1602.

The base receiver 1610, in turn, can connect the mobile device 1602 to the Internet 1620 over a packet switched link. The internet 1620 can support application services and service layers for distributing data from the measurement system 1655 to the mobile device 1602. The mobile device 1602 can also connect to other communication devices through the Internet 1620 using a wireless communication channel.

The mobile device 1602 can also connect to the Internet 1620 over the WLAN 1605. Wireless Local Access Networks (WLANs) provide wireless access within a local geographical area. WLANs are typically composed of a cluster of Access Points (APs) 1604 also known as base stations. The measurement system 1655 can communicate with other WLAN stations such as laptop 1603 within the base station area. In typical WLAN implementations, the physical layer uses a variety of technologies such as 802.11b or 802.11g WLAN technologies. The physical layer may use infrared, frequency hopping spread spectrum in the 2.4 GHz Band, direct sequence spread spectrum in the 2.4 GHz Band, or other access technologies, for example, in the 5.8 GHz ISM band or higher ISM bands (e.g., 24 GHz, etc).

By way of the communication network 1600, the measurement system 1655 can establish connections with a remote server 1630 on the network and with other mobile devices for exchanging data. The remote server 1630 can have access to a database 1640 that is stored locally or remotely and which can contain application specific data. The remote server 1630 can also host application services directly, or over the internet 1620.

It should be noted that very little data exists on implanted orthopedic devices. Most of the data is empirically obtained by analyzing orthopedic devices that have been used in a human subject or simulated use. Wear patterns, material issues, and failure mechanisms are studied. Although information can be garnered through this type of empirical study, it does not yield substantive data about the initial installation, post-operative use, and long term use from a measurement perspective. Just as each person is different, each device installation is different having variations in initial loading, balance, and alignment. Having measured data and using the data to install an orthopedic device will greatly increase the consistency of the implant procedure thereby reducing rework and maximizing the life of the device. In at least one example embodiment, the measured data can be collected to a database where it can be stored and analyzed. For example, once a relevant sample of the measured data is collected, it can be used to define optimal initial measured settings, geometries, and alignments for maximizing the life and usability of an implanted orthopedic device.

FIG. 17 depicts a diagrammatic representation of a machine in the form of a computer system 1700 within which a set of instructions, when executed, may cause the machine to perform any one or more of the methodologies discussed above. In some embodiments, the machine operates as a standalone device. In some embodiments, the machine may be connected (e.g., using a network) to other machines. In a networked deployment, the machine may operate in the capacity of a server or a client user machine in server-client user network environment, or as a peer machine in a peer-to-peer (or distributed) network environment.

The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. It will be understood that a device of the present disclosure includes broadly any electronic device that provides voice, video or data communication. Further, while a single machine is illustrated, the term “machine” shall also be taken to include any collection of machines that individually or jointly execute a set (or multiple sets) of instructions to perform any one or more of the methodologies discussed herein.

The computer system 1700 may include a processor 1702 (e.g., a central processing unit (CPU), a graphics processing unit (GPU, or both), a main memory 1704 and a static memory 1706, which communicate with each other via a bus 1708. The computer system 1700 may further include a video display unit 1710 (e.g., a liquid crystal display (LCD), a flat panel, a solid state display, or a cathode ray tube (CRT)). The computer system 1700 may include an input device 1712 (e.g., a keyboard), a cursor control device 1714 (e.g., a mouse), a disk drive unit 1716, a signal generation device 1718 (e.g., a speaker or remote control) and a network interface device 1720.

The disk drive unit 1716 can be other types of memory such as flash memory and may include a machine-readable medium 1722 on which is stored one or more sets of instructions (e.g., software 1724) embodying any one or more of the methodologies or functions described herein, including those methods illustrated above. The instructions 1724 may also reside, completely or at least partially, within the main memory 1704, the static memory 1706, and/or within the processor 1702 during execution thereof by the computer system 1700. The main memory 1704 and the processor 1702 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine readable medium containing instructions 1724, or that which receives and executes instructions 1724 from a propagated signal so that a device connected to a network environment 1726 can send or receive voice, video or data, and to communicate over the network 1726 using the instructions 1724. The instructions 1724 may further be transmitted or received over a network 1726 via the network interface device 1720.

While the machine-readable medium 1722 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present disclosure.

The term “machine-readable medium” shall accordingly be taken to include, but not be limited to: solid-state memories such as a memory card or other package that houses one or more read-only (non-volatile) memories, random access memories, or other re-writable (volatile) memories; magneto-optical or optical media such as a disk or tape; and carrier wave signals such as a signal embodying computer instructions in a transmission medium; and/or a digital file attachment to e-mail or other self-contained information archive or set of archives is considered a distribution medium equivalent to a tangible storage medium. Accordingly, the disclosure is considered to include any one or more of a machine-readable medium or a distribution medium, as listed herein and including art-recognized equivalents and successor media, in which the software implementations herein are stored.

Although the present specification describes components and functions implemented in the embodiments with reference to particular standards and protocols, the disclosure is not limited to such standards and protocols. Each of the standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same functions are considered equivalents.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

In general, artificial components for other joint replacement surgeries have a similar operational form as the knee joint example. The joint typically comprises two or more bones with a cartilaginous surface as an articular surface that allows joint movement. The cartilage also acts to absorb loading on the joint and prevents bone-to-bone contact. Reconstruction of the hip, spine, shoulder, and other joints has similar functioning insert structures having at least one articular surface. Like the knee joint, these other insert structures typically comprise a polymer material. The polymer material is formed for a particular joint structure. For example, the hip insert is formed in a cup shape that is fitted into the pelvis. In general, the size and thickness of these other joint inserts allow the integration of the sensing module. It should be noted that the sensing module disclosed herein contemplates use in both trial inserts and permanent inserts for the other joints of the muscular-skeletal system thereby providing quantitative parameter measurements during and post surgery.

While the present invention has been described with reference to particular embodiments, those skilled in the art will recognize that many changes may be made thereto without departing from the spirit and scope of the present invention. Each of these embodiments and obvious variations thereof is contemplated as falling within the spirit and scope of the invention.

What is claimed is:

1. A measurement system for measuring a parameter of the muscular-skeletal system comprising a prosthetic knee insert that includes:
at least one sensor;
electronic circuitry operatively coupled to the at least one sensor; and
a power source coupled to the electronic circuitry where the electronic circuitry and the power source are located within the prosthetic knee insert in an unloaded or lightly loaded region therein.

2. The measurement system of claim 1 where the prosthetic knee insert includes a first articular surface and a second articular surface for allowing movement of the muscular-skeletal system where the electronic circuitry and the power source are located between the first and second articular surfaces.

3. The measurement system of claim 2 where the electronic circuitry comprises one or more electrical components mounted and coupled to form a circuit on a printed circuit board.

4. The measurement system of claim 3 further including an application specific integrated circuit mounted to the printed circuit board to reduce form factor and power dissipation.

5. The measurement system of claim 4 where the power source is mounted to the printed circuit board and where a clip overlies the power source and couples to a slot in the at least one retaining feature.

6. The measurement system of claim 3 further including an interior surface within the insert where at least one retaining feature extends from the interior surface for retaining the printed circuit board.

7. The measurement system of claim 4 further including at least one channel adjacent to the interior surface for routing interconnect to the printed circuit board.

8. The measurement system of claim 2 where the prosthetic knee insert is substantially equal dimensionally to a final knee insert.

9. The measurement system of claim 8 further including:
at least three sensors underlying the first articular surface; and
at least three sensors underlying the second articular surface.

10. The measurement system of claim 9 further including:
a first load plate coupled to the first articular surface for transferring a load applied to the first articular surface to the at least three sensors underlying the first articular surface; and
a second load plate coupled to the second articular surface for transferring a load applied to the second articular surface to the at least three sensors underlying the second articular surface.

11. A prosthetic knee insert for measuring a force, pressure, or load applied by the muscular-skeletal system comprising:
a first articular surface;
a second articular surface; and
a region between the first and second articular surfaces that is unloaded or lightly loaded by the muscular-skeletal region; and
a cavity within the insert underlying the region for housing electronic circuitry for measuring the force, pressure, or load within the insert.

12. The prosthetic knee insert of claim 11 further including:
at least three sensors underlying the first articular surface; and
at least three sensors underlying the second articular surface where the electronic circuitry is operatively coupled to each of the at least three sensors underlying the first and second articular surfaces.

13. The prosthetic knee insert of claim 12 further including:
a first load plate coupled between the at least three sensors and the first articular surface; and
a second load plate coupled between the at least three sensors and the second articular surface.

14. The prosthetic knee insert of claim 11 where at least one retaining feature extends from an interior surface of the cavity for retaining the electronic circuitry within the cavity.

15. The prosthetic knee insert of claim 14 further including at least one channel adjacent to the interior surface for routing interconnect from the at least three sensors underlying the first and second articular surfaces to the electronic circuitry.

16. A method of isolating electronic circuitry for measuring a parameter of the muscular-skeletal system in a prosthetic insert component comprising the steps of:
forming an enclosure having at least one articular surface and a load bearing surface where a force, pressure, or load is applied by the muscular-skeletal system to the articular and load bearing surfaces;
placing the electronic circuitry in an un-loaded or lightly loaded region within the enclosure where the insert is substantially equal dimensionally to a final insert; and
sealing the enclosure to isolate the electronic circuitry from an external environment.

17. The method of claim 16 further including the steps:
providing a first support structure having the at least one articular surface and a surface that is un-loaded or lightly loaded;
providing a second support structure having the load bearing surface; and
coupling the first and second support structures together such that the electronic circuitry is located underlying the un-loaded or lightly loaded surface of the first support structure.

18. The method of claim 16 further including a step of retaining the electronic circuitry by one or more retaining features within the enclosure.

19. The method of claim 16 further including a step of coupling a plurality of sensors between the articular surface and the load bearing surface of the enclosure to measure a force, pressure, or load applied thereacross.

20. The method of claim 16 further including a step of disposing of the insert after using the insert intra-operatively.
